(19) United States
(12) Patent Application Publication
(10) Pub. No.: US 2015/0197729 A1
(43) Pub. Date: Jul. 16, 2015

(54) Compositions and Methods for Regulating Cytochrome c-Mediated Apoptosis by tRNA

(71) Applicant: The Trustees of the University of Pennsylvania, Philadelphia, PA (US)

(72) Inventors: Xiaolu Yang, Wynnewood, PA (US); Yide Mei, Philadelphia, PA (US); Jeongsik Yong, Broomall, PA (US)

(21) Appl. No.: 14/636,516

(22) Filed: Mar. 3, 2015

Related U.S. Application Data
(63) Continuation of application No. 13/500,271, filed on Jun. 18, 2012, now abandoned.
(60) Provisional application No. 61/249,115, filed on Oct. 6, 2009.

(51) Int. Cl. C12N 5/09; A61K 38/02; A61K 38/43; C12N 5/071

(52) U.S. Cl. CPC C12N 5/0693; C12N 5/0602; A61K 38/02; A61K 38/43; C12N 2501/06; C12N 2501/73

(57) ABSTRACT

The invention relates to the discovery that tRNA is a potent regulator of cell survival. tRNA regulates the interaction between cytochrome c and Apaf-1 and subsequently Apaf-1 oligomerization into an apoptosome which in turn recruits and oligomerizes the caspase cascade which ultimately leads to cell death. Accordingly, the present invention provides compositions and methods for regulating cell survival.

### BACKGROUND OF THE INVENTION

Apoptosis plays an essential role in development, maintaining homeostasis, and protection against viral infection and cancer (Thompson, 1995; Vaux and Korsmeyer, 1999). This stereotypic form of programmed cell death, characterized by a set of morphological and biochemical changes, is executed by caspases (Chang and Yang, 2000; Degterev et al, 2003; Thornberry and Lazebnik, 1998). Two major apoptosis pathways exist in mammalian cells; they are triggered by cell-intrinsic and extrinsic stimuli, respectively. The intrinsic pathway, which is activated in part by developmental lineage information, oncogene activation, DNA damage, and nutrition deprivation, is defined by the release of mitochondrial cytochrome c into the cytosol. In the cytosol, cytochrome c binds to Apaf-1, enabling Apaf-1 to assemble into an oligomeric complex known as the apoptosome. The apoptosome then recruits and oligomerizes the precursor of an initiator caspase, caspase-9, leading to its auto-proteolytic activation. This is followed by trans-activation of down-stream effector caspases such as caspase-3 and 7 by caspase-9, cleavage of various cellular proteins by effector caspases, and ultimately cell death (Riedl and Salvesen, 2007; Wang, 2001; FIG. 6). The assembly of the apoptosome requires the hydrolysis of an Apaf-1-bound dATP to dADP and the subsequent exchange of the dADP with a free dATP (Chandra et al., 2006; Kim et al., 2005; Liu et al., 1996; Riedl et al., 2005). The hydrolysis of dATP is enhanced by the combined action of at least three proteins: the tumor suppressor PHAPI, cellular apoptosis susceptibility protein (CAS), and heat shock protein 70 (Hsp70) (Kim et al., 2008). In contrast, apoptosome formation is inhibited by HSP27, HSP90, and the oncoprotein prothymosin-a (ProT), as well as the cations potassium and calcium (Riedl and Salvesen, 2007; Schafer and Kornbluth, 2006). Additionally, although low levels of dATP promote apoptosome formation, high levels of dATP inhibit it (Chandra et al., 2006). However, the role of the polymer ribonucleotide (RNA) in caspase-9 activation has not been established.

tRNA has a fundamental role in protein synthesis, as it provides the link between a genetic codon and an amino acid (Hopper and Shaheen, 2008; Ibba et al., 2000; Weisblum, 1999). For each amino acid there is at least one tRNA, which is coupled to the amino acid through an aminoacyl-tRNA transferase. The tRNA then delivers the amino acid to the ribosome for incorporation into a polypeptide through the interaction between the anti-codon in tRNA and a codon in mRNA. In addition to a role in translation, some tRNAs also serve as primers for reverse transcription to make DNA out of RNA genome. The function of tRNAs beyond the transmission of genetic information is not clear. New compounds and methods of regulating apoptosis would be useful tools for the treatment of a variety of diseases and disorders including tumor therapy. The instant invention meets this need.

### SUMMARY OF THE INVENTION

The present invention provides a method of enhancing survival of a cell. The method comprises inhibiting the formation of an apoptosome in a cell by contacting the cell with an effective amount of a tRNA activator, wherein when the tRNA activator contacts the cell, the tRNA activator increases the expression, function, stability, or activity of the tRNA, wherein the tRNA binds to cytochrome c, thereby enhancing survival of the cell.

In one embodiment, the cell is a mammalian cell. Preferably, the mammalian cell is a human cell.

The invention also provides a method of inhibiting survival of a cell. The method comprises enhancing formation of an apoptosome in a cell by contacting the cell with an effective amount of a tRNA inhibitor, wherein when the tRNA inhibitor contacts the cell, the tRNA inhibitor decreases expression, function, stability, or activity of the tRNA, wherein the tRNA does not bind to cytochrome c, thereby inhibiting survival of the cell.

In one embodiment, the tRNA inhibitor is selected from the group consisting of a protein, a peptide, an siRNA, a ribozyme, an antisense, an aptamer, a peptidomimetic, a small molecule, or any combination thereof.

In one embodiment, the cell is a mammalian cell, preferably a human cell, more preferably a human cancer cell.

In one embodiment, the protein is an RNase. Preferably, the RNase is onconase.

In one embodiment, the tRNA inhibitor is administered in combination with a therapeutically effective amount of another therapeutic agent. In some instances, the therapeutic agent is doxorubicin.

The invention also provides a method of augmenting tRNA expression, function or activity in a cell. The method comprising contacting the cell with a tRNA activator, wherein when the tRNA activator contacts the cell, the tRNA activator augments the tRNA expression, function, or activity in the cell, wherein the tRNA does not bind to cytochrome c, thereby inhibiting survival of the cell.

The invention also provides a method of inhibiting tRNA expression, function or activity in a cell. The method comprising contacting a cell with a tRNA inhibitor, wherein when the tRNA inhibitor contacts the cell, the tRNA inhibitor reduces the tRNA expression, function, or activity in the cell, wherein the tRNA does not bind cytochrome c, thereby inhibiting survival of the cell.

The invention also provides a method of inhibiting an interaction between cytochrome c and Apaf-1 in a cell. The method comprising contacting the cell with an effective amount of a tRNA activator, wherein the tRNA activator increases tRNA expression, activity, stability, or function in the cell, thereby inhibiting the interaction between cytochrome c and Apaf-1 and enhancing cell survival.

The invention also provides a method of increasing an interaction between cytochrome c and Apaf-1 in a cell. The method comprising contacting the cell with an effective amount of a tRNA inhibitor, wherein the tRNA inhibitor increases tRNA expression, activity, stability, or function in the cell, thereby increasing the interaction between cytochrome c and Apaf-1, thereby decreasing cell survival.

The invention also provides a method of treating a disease associated with aberrant cytochrome c release in a mammal. The method comprises administering to a mammal in need thereof a composition comprising a tRNA activator or a tRNA inhibitor.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1F, is a series of images depicting RNase A enhancement of cytochrome c-induced caspase-9 activation while total cellular RNA inhibits it. FIG. 1A and FIG. 1B depict the effect of incubating HeLa S100 with cytochrome c (20 μg/ml) in the absence (−) or presence of increasing amounts of RNase A. Extracts were immunoblotted with anti-caspase-9, anti-caspase-3, and anti-actin antibodies. C9, procaspase-9; C3, procaspase-3. Molecular weight 30 (MW) markers (in kilodaltons) are indicated on the left. FIG. 1B depicts RNA separated via PAGE under denaturing conditions and stained with ethidium bromide. FIG. 1C depicts activation of caspase-9 and -3 in HeLa S100 extracts treated with cytochrome c (20 pg/ml), RNase A (30 ng/ml), and RNase inhibitor (In). FIG. 1D depicts reticulocyte lysates containing in vitro-translated, ³⁵S-labeled procaspase-9 treated without or with cytochrome c (200 μg/ml), in the presence of the indicated concentration of RNase A. Caspase-9 was detected by autoradiography. FIG. 1E depicts activation of caspase-9 and -3 in Jurkat S100 extracts treated with cytochrome c alone or in combination with RNA. FIG. 1F depicts in vitro-translated, 35S-labeled procaspase-9 incubated with purified full-length Apaf-1 (10 nM), dATP (1 mM), cytochrome c (20 pg/ml), and increasing amounts of total cellular RNA (0.1, 0.2, and 0.4 μg/μl). Caspase-9 was detected by autoradiography.

FIG. 2, comprising FIG. 2A through FIG. 2E, depicts RNA interference with cytochrome c:Apaf-1 interaction and inhibition of apoptosome formation. FIG. 2A depicts Jurkat S100 extracts incubated alone (−), with cytochrome c, or with cytochrome c plus RNA. Extracts were fractionated on a Superose 6 gel filtration column, and the fractions were analyzed by Western blot. The positions of molecular weight standards (in kilodaltons) for the column are marked at the top. FIG. 2B depicts His-tagged Apaf-1 bound to Ni-NTA beads was incubated with or without cytochrome c, or with cytochrome c plus increasing amounts of RNA (0.1, 0.2, and 0.4 μg/μl). One percent of the input and bead-bound proteins were analyzed by Western blot analysis using anti-cytochrome c or anti-Apaf-1 antibodies as indicated. FIG. 2C depicts in vitro-translated, ³⁵S-labeled procaspase-9 was incubated with purified Apaf-1 (1-591), dATP, and increasing amount of RNA as indicated, at 30° C. for 1 h. FIG. 2D depicts Jurkat S100 extracts incubated with cytochrome c for the indicated durations before being analyzed for caspase-9 activation. FIG. 2E depicts Jurkat S100 extracts with cytochrome c for a period before the addition of tRNA at the indicated time (lanes 3-8). The total reaction time for each sample was 2 h. Activation of caspase-9 was analyzed by Western blot.

FIG. 3, comprising FIG. 3A through FIG. 3E, is a series of images depicting the interaction of cytochrome c with tRNA in vivo and in vitro and the effect of tRNA on caspase-9 activation. FIG. 3A depicts RNA in the immunoprecipitates analyzed by Northern blotting using indicated, radiolabeled mitochondrial and cytosolic tRNAs, SS rRNA, and Ul snRNA. Input samples contained −1% of the RNA used for IP. FIG. 3B depicts cytochrome c and Smac in immunoprecipitates and −1.5% of the input and were analyzed by Western blot. *, Smac precursor. FIG. 3C depicts in vitro synthesis of, [³²P]UTP-labeled tRNA were incubated with increasing amounts (0.5, 2.5, 12.5 FAM) of cytochrome c for 45 min at 30° C. Reaction mixtures were incubated with 0.5 M Urea (final concentration) for 10 min and analyzed by 6% native gel electrophoresis and autoradiography. Cytochrome c:tRNA complexes are indicated. F.P., free tRNA probes. FIG. 3D depicts Jurkat S100 extracts incubated with cytochrome c and increasing amounts of total RNA, rRNA, tRNA (0.1, 0.2, and 0.4 μg/R1), and mRNA (0.02, 0.04, 0.08 μg/μl) at 37° C. for 1 h. Activation of caspase-9 was analyzed by Western blot. Less amounts of mRNA were used because in cells it is expressed at much lower levels compared with either tRNA or rRNA. FIG. 3E depicts Jurkat S100 extracts incubated with increasing concentrations of cytochrome c in the absence or presence of different concentrations of tRNA. After 1 h incubation, Western blotting was performed using anti-caspase-9 and -3 antibodies.

FIG. 4, comprising FIG. 4A and FIG. 4B, depicts the effect of microinjection of tRNA on cytochrome c-induced apoptosis. FIG. 4A depicts representative images of injected cells. Arrowheads indicate apoptotic cells. FIG. 4B depicts the percentage of apoptosis of injected cells. Data shown are means and standard deviations of three independent experiments.

FIG. 5, comprising FIG. 5A through FIG. 5D, depicts the effect of degradation of tRNA on apoptosis via the intrinsic pathway. Figure SA depicts HeLa cells transfected with 1 pg/ml onconase and cultured for the indicated period of time. Left: total RNA extracted with Trizol reagents separated by 8% urea containing PAGE and visualized by ethidium bromide staining Right: cell extracts analyzed for the activation of caspase-9 and caspase-3 and the cleavage of PARP by Western blot. The levels of actin are shown as a loading control. FIG. 5B depicts Apaf-1^{±/±} and Apaf-1⁴⁻ MEF cells treated with indicated amount of onconase for 24 h. Top: percentages of apoptosis (means and standard deviations) of three independent experiments are shown. Bottom: cell lysates were analyzed by Western blot for Apaf-1 expression and PARP cleavage. FIG. 5C depicts HeLa cells transfected with indicated amount of onconase. Starting 3 h post-transfection, the cells were incubated with or without doxorubicin (Dox, 1 μg/ml) for an additional 12 h. Left: percentages of apoptosis (means and standard deviations) of three independent experiments are shown. Right: the activation of caspase-9 and -3 and the processing of PARP were examined by Western blot. FIG. 5D depicts HeLa cells transfected with or without onconase (1 μg/ml). 3 h after transfection, cells were treated with Dox (1 μg/ml) for another 12 h. S100 extracts were fractionated on a Superose 6 gel filtration column, and fractions were analyzed by Western blot using anti-Apaf-1 antibody.

FIG. 6 depicts the intrinsic apoptosis pathway. Various intracellular apoptotic stimuli provoke the release of cytochrome c (cyt. c) from mitochondria (a). In the cytosol, cytochrome c binds to Apaf-1, promoting the hydrolysis of Apaf-1-bound dATP to dADP. This is followed by the release of dADP in exchange for dATP and the assembly of Apaf-1 into a heptameric complex known as the apoptosome (b, only two molecules of Apaf-1 are shown). The apoptosome recruits and oligomerizes procaspase-9 (Pro-C9) (c), leading to the auto-proteolytic processing of procaspase-9 to the mature caspase-9 (d). Mature caspase-9 then converts procaspase-3 (pro-C3), which pre-exists as a dimer, to the active form (e).

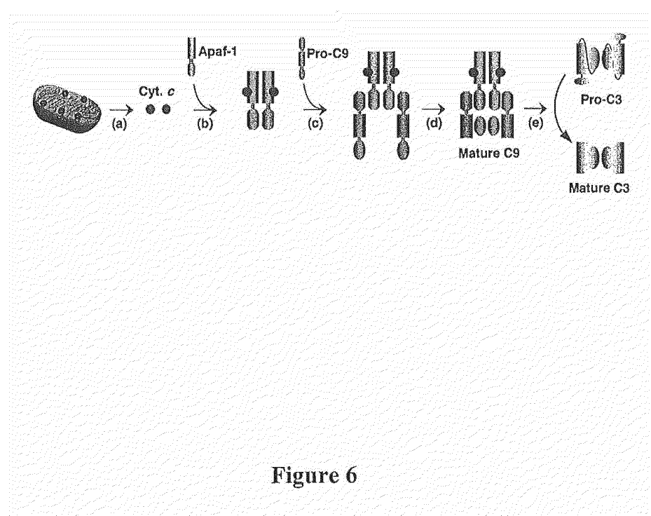

FIG. 7 depicts Cytochrome c induces caspase-9 and caspase-3 activation in Jurkat S100 extract. Jurkat S100 extracts were incubated with cytochrome c (cyt. c) (20 μg/ml) at 37° C. for the indicated time periods. The activation of caspase-9 and -3 in the extracts was analyzed by Western blots. The amount of actin in the extracts is shown for equivalent sample loading. Molecular weight standards (in kDa) are marked on the left.

FIG. 8 depicts the specitifity of the cytochrome c association with tRNA. The anti-cytochrome c, anti-Smac, and control immunoprecipitates (FIG. 3A, B) were analyzed by Northern blotting using radiolabeled probes for 7SK RNA, 7SL RNA, RNase MRP and P RNAs, human vault RNA (hvg3), and human Y1 RNA (hY1). Input samples contained ˜1% of the RNA used for IP.

FIG. 9 depicts tRNA block of apoptosome formation and caspase-9 activation. Jurkat S100 extracts were incubated without (ctrl) or with cytochrome c, or with cytochrome c plus tRNA. The extracts were resolved on Superose 6 gel filtration column, and fractions were analyzed by Western blot. The molecular weight standards for the column are marked at the top.

FIG. 10, comprised of FIG. 10A and FIG. 10B, depicts Onconase treatment enhances cytochrome c-induced caspase-9 activation in S100 extract. HeLa S100 extracts were pre-incubated with indicated amounts of onconase (Onc) for 20 min incubation at room temperature. (A) RNA was isolated and analyzed by denaturing PAGE and stained with ethidium bromide. (B) Extracts were further incubated with cytochrome c (20 μg/ml) at 37° C. for an additional 1 h, and then immunoblotted with anti-caspase-9, anti-caspase-3, and anti-actin antibodies.

FIG. 11 depicts tRNA degradation in onconase and doxorubincin-treated cells. Total RNAs from the HeLa cells treated with onconase and/or doxorubicin (FIG. 5C) were extracted using Trizol reagents, fractionated by 8% urea-containing PAGE, and visualized by ethidium bromide staining. Note that onconase-treated cells exhibited less apoptosis (FIG. 5C) but more dramatic decrease in tRNA compared with doxorubicin-treated cells, indicating that the degradation of tRNA in onconase-treated cells is not a secondary effect of apoptosis. The decrease of tRNA in doxorubicin-treated cells and the further decrease of tRNA in the cells treated with onconase plus doxorubicin were likely due to cell death.

FIG. 12, comprising FIGS. 12A and 12B, is a series of images depicting cytochrome c and tRNA interaction affinity. FIG. 12A is an image demonstrating that Cy3-labeled tRNA_{Cys} is with increasing cytochrome c. FIG. 12B is an image depicting hyperbolic fit of fluorescence quenching of K_{d}=3.5 μM.

FIG. 13, comprising FIGS. 13A and 13B, is a series of images depicting characteristics of cytochrome c and tRNA interaction. FIG. 13A is an image depicting binding of increasing concentrations of tRNA to immobilized cytochrome c in low salt conditions by surface plasmon resonance. FIG. 13B is an image depicting tRNA, but not ribosomal RNA or a DNA oligo of a phenylalanine tRNA sequence binding to immobilized cytochrome c at physiologic salt concentration.

FIG. 14 is a schematic depicting strategy for in vivo capture of ribonucleoparticles and their analysis by high-throughput sequencing.

FIG. 15 is an image depicting distribution of RNA sequences recovered after immunoprecipitation with cytochrome c or gemin5 antibodies, followed by Rnase digestion, reverse transcription and sequencing.

FIG. 16, comprising FIGS. 16A through 16C, is a series of images demonstrating cytochrome c oxidation. FIG. 16A is an image of a Western blot showing relative enrichment of cytochrome c oxidase in mitochondrial fractions (M). FIG. 16B is an image demonstrating that addition of crude mitochondrial extract causes oxidation of cytochrome c which is followed as optical density at 550 nm. FIG. 16C is an image demonstrating that tRNA addition inhibits the rate of cytochrome c oxidation.

### DETAILED DESCRIPTION OF THE INVENTION

The invention relates to the discovery that tRNA is a potent regulator of cell survival. tRNA regulates the interaction between cytochrome c and Apaf-1 and subsequently Apaf-1 oligomerization into an apoptosome which in turn recruits and oligomerizes the caspase cascade which ultimately leads to cell death. Accordingly, the present invention provides compositions and methods for regulating cell survival.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art 20 and will vary to some extent on the context in which it is used.

The phrase “activator,” as used herein, means to increase a tRNA's expression, stability, function or activity by a measurable amount. Activators are compounds that, e.g., partially or totally stimulate, increase, promote, increase activation, activate, sensitize, or up-regulate, a tRNA's stability, expression, function and activity.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator,” as the term is used herein, is meant any device including, but not limited to, a hypodermic syringe, a pipette, and the like, for 5 administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease, or disorder, the frequency with which such a symptom is experienced by a patient, or both, are reduced.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The terms “neoplasia,” “hyperplasia,” and “tumor” are often commonly referred to as “cancer,” which is a general name for more than 100 disease that are characterized by uncontrolled, abnormal growth of cells. Examples of malignancies include but are not limited to acute lymphoblastic leukemia; acute myeloid leukemia; adrenocortical carcinoma; AIDS-related lymphoma; cancer of the bile duct; bladder cancer; bone cancer, osteosarcomal malignant fibrous histiocytomal brain stem gliomal brain tumor; breast cancer, bronchial adenomas; carcinoid tumors; adrenocortical carcinoma; central nervous system lymphoma; cancer of the sinus, cancer of the gall bladder; gastric cancer; cancer of the salivary glands; cancer of the esophagus; neural cell cancer; intestinal cancer (e.g., of the large or small intestine); cervical cancer; colon cancer, colorectal cancer; cutaneous T-cell lymphoma; B-cell lymphoma; T-cell lymphoma; endometrial cancer; epithelial cancer; endometrial cancer; intraocular melanoma; retinoblastoma; hairy cell leukemia; liver cancer; Hodgkin's disease; Kaposi's sarcoma; acute lymphoblastic leukemia; lung cancer; non-Hodgkin's lymphoma; melanoma; multiple myeloma; neuroblastoma; prostate cancer; retinoblastoma; Ewing's sarcoma; vaginal cancer; Waldenstrom's macroglobulinemia; adenocarcinomas; ovarian cancer, chronic lymphocytic leukemia, pancreatic cancer, and Wilm's tumor.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing nonpeptidic structural elements that is capable of mimicking the biological action of a parent 10 peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

By the term “specifically binds,” as used herein, is meant a molecule, such as an antibody, which recognizes and binds to another molecule or feature, but does not substantially recognize or bind other molecules or features in a sample. As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

The phrase “inhibit,” as used herein, means to reduce a tRNA's expression, stability, function or activity by a measurable amount or to prevent entirely. Inhibitors are compounds that, e.g., partially or totally block stimulation, decrease, prevent, delay activation, inactivate, desensitize, or down regulate a tRNA's stability, expression, function and activity, e.g., antagonists.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

It is understood that any and all whole or partial integers between any ranges set forth herein are included herein.

### Description:

The present invention is based on the discovery that tRNA prevents apoptosis by binding to cytochrome c and blocking the interaction of cytochrome c with Apaf-1 thereby abrogating Apaf-1 oligomerization and the formation of an apoptosome as well as subsequent caspase activation and inhibiting apoptosis. Accordingly, the instant invention describes compositions and methods for regulating cell survival by regulating tRNA expression in a cell.

In one embodiment, the instant invention includes a method of enhancing cell survival where the method includes activating a tRNA molecule in a cell, thereby inhibiting the interaction of cytochrome c with Apaf-1, abrogating Apaf-1 oligomerization, and subsequent caspase activation and preventing cell death.

In another embodiment, the instant invention includes a method of reducing cell survival where the method includes inhibiting the expression of a tRNA molecule in a cell, thereby enhancing the interaction of cytochrome c with Apaf-1, Apaf-1 oligomerization, and subsequent caspase activation and increasing apoptosis.

In another embodiment, the instant invention includes a method of inhibiting apoptosis in a cell, the method including activating a tRNA molecule in a cell, thereby blocking the interaction of cytochrome c with Apaf-1, abrogating Apaf-1 oligomerization, and subsequent caspase activation and inhibiting apoptosis in the cell.

In another embodiment, the instant invention includes a method of inducing apoptosis in a cell, the method includes inhibiting a tRNA molecule in a cell, thereby augmenting the interaction of cytochrome c with Apaf-1, Apaf-1 oligomerization, and subsequent caspase activation and enhancing apoptosis in the cell. In one aspect the cell is a cancer cell. The instant invention further includes a method of regulating the interaction of cytochrome c with Apaf-1 where the method includes regulating tRNA expression, activity or function in a cell. In one embodiment, the invention includes a method of increasing the interaction of cytochrome c with Apaf-1 where the method includes inhibiting tRNA expression, activity or function in a cell. In another embodiment, the invention includes a method of inhibiting the interaction of cytochrome c with Apaf-1 where the method includes enhancing tRNA expression, activity, or function in a cell.

The present invention provides compositions and methods for regulating the expression, function, or activity of a tRNA in a cell in a subject. In one embodiment, a tRNA regulator is a tRNA activator, where the tRNA activator augments or enhances the expression, function, stability, or activity of a tRNA in cell cell. The methods include administering to a subject in need thereof a therapeutically effective amount of a tRNA activator, where a tRNA activator is a compound, molecule, or composition that increases tRNA expression, function, stability or activity in a cell, where the enhancer augments the expression, function, or activity of a tRNA in the cell. Increasing or augmenting tRNA activity can be accomplished using any method known to the skilled artisan. Examples of methods to increase tRNA activity include, but are not limited to increasing expression of an endogenous tRNA gene, increasing expression of tRNA mRNA, and increasing activity of tRNA. A tRNA activator may therefore be a compound or composition that increases expression of a tRNA gene, a compound or composition that increases tRNA half-life, stability and/or expression, or a compound or composition that increases tRNA function. A tRNA activator may be any type of compound, including but not limited to, a polypeptide, a nucleic acid, an aptamer, a peptidometic, and a small molecule, or combinations thereof.

In another embodiment, the tRNA regulator is a tRNA inhibitor where the tRNA inhibitor inhibits or reduces the expression, function, stability, or activity of a tRNA in a cell. The methods include administering to a subject in need thereof a therapeutically effective amount of a tRNA inhibitor, where the tRNA inhibitor is a compound, molecule, or composition that inhibits tRNA expression, function, stability, or activity in a cell, where the enhancer augments the expression, function, or activity of a tRNA in the cell. Inhibiting tRNA activity can be accomplished using any method known to the skilled artisan. Examples of methods to inhibit tRNA activity include, but are not limited to decreasing expression of an endogenous tRNA gene, decreasing expression of tRNA mRNA, and inhibiting activity of tRNA. A tRNA inhibitor may therefore be a compound or composition that decreases expression of a tRNA gene, a compound or composition that decreases tRNA half-life, stability and/or expression, or a compound or composition that inhibits tRNA function. A tRNA inhibitor may be any type of compound, including but not limited to, a polypeptide, a nucleic acid, an aptamer, a peptidometic, and a small molecule, or combinations thereof. A tRNA inhibitor may also be a protein, including an enzyme, such an an RNase. In one embodiment, the tRNA regulator is onconase.

In one embodiment of the invention, a subject is a mammal. In another embodiment, the mammal is a human. tRNA expression, function or activity may be regulated directly or indirectly. For example, tRNA expression, function, or activity may be directly regulated by compounds or compositions that directly interact with tRNA. such as ocogenase which hydrolyzes tRNA. Alternatively, tRNA expression, function, or activity may be inhibited indirectly by compounds or compositions that affect the expression, function, or activity of tRNA, its downstream effectors, or its upstream regulators.

Inhibiting tRNA expression, function, or its activity may be accomplished by any means known in the art or as described herein. Enhancing or increasing tRNA expression, function, or activity can be accomplished using any method known to the skilled artisan. Examples of methods to enhance or increase tRNA expression include, but are not limited to increasing expression of an endogenous tRNA gene. An agent, composition or compound that enhances or increases tRNA expression or activity may be a compound or composition that increases expression of a tRNA gene, a compound or composition that increases tRNA half-life, stability and/or expression, or a compound or composition that enhances tRNA function. An agent, composition or compound that enhances or increases tRNA expression, function, or activity may be any type of compound, including but not limited to, a polypeptide, a nucleic acid, an aptamer, a peptidometic, and a small molecule, or combinations thereof.

The present invention should in no way be construed to be limited to the inhibitors or activators described herein, but rather should be construed to encompass any activator or inhibitor of the tRNA system in intrinsic apoptosis, both known and unknown, that regulates cell survival.

### Compositions:

tRNA:

Transfer RNA (abbreviated tRNA) is a small RNA molecule (usually about 74-95 nucleotides) that transfers a specific active amino acid to a growing polypeptide chain at the ribosomal site of protein synthesis during translation. It has a 3′ terminal site for amino acid attachment. This covalent linkage is catalyzed by an aminoacyl tRNA synthetase. It also contains a three base region called the anticodon that can base pair to the corresponding three base codon region on mRNA. Each type of tRNA molecule can be attached to only one type of amino acid, but because the genetic code contains multiple codons that specify the same amino acid, tRNA molecules bearing different anticodons may also carry the same amino acid.

An anticodon is a unit made up of three nucleotides that correspond to the three bases of the codon on the mRNA. Each tRNA contains a specific anticodon triplet sequence that can base-pair to one or more codons for an amino acid. For example, one codon for lysine is AAA; the anticodon of a lysine tRNA might be UUU. Some anticodons can pair with more than one codon due to a phenomenon known as wobble base pairing. Frequently, the first nucleotide of the anticodon is one of two not found on mRNA: inosine and pseudouridine, which can hydrogen bond to more than one base in the corresponding codon position. In the genetic code, it is common for a single amino acid to be specified by all four third-position possibilities, or at least by both Pyrimidines and Purines; for example, the amino acid glycine is coded for by the codon sequences GGU, GGC, GGA, and GGG.

The instant invention further includes tRNA-like molecules which have the same specificity, activity and function as tRNA.

1 Inhibitors of tRNA Expression, Function, or Activity

a. Antisense Nucleic Acids

In one embodiment of the invention, an antisense nucleic acid sequence which is expressed by a plasmid vector is used to inhibit tRNA expression. The antisense expressing vector is used to transfect a mammalian cell or the mammal itself, thereby causing reduced endogenous expression of a tRNA, or a regulator thereof.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to 20 about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

b. Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit tRNA expression. Ribozymes useful for inhibiting the expression of a target molecule may be designed by incorporating target sequences into the basic ribozyme structure which are complementary. Ribozymes targeting tRNA, may be synthesized using commercially available reagents (Applied Biosystems, Inc., Foster City, Calif.) or they may be genetically expressed from DNA encoding them.

c. siRNA

In one embodiment, siRNA is used to decrease the level of tRNA. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of tRNA using RNAi technology.

i. Modification of siRNA

Following the generation of the siRNA polynucleotide of the present invention, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide of the invention may be further modified to increase its stability in vivo. Possible modifications include, but are not limited to, the addition of flanking sequences at the 5′ and/or 3′ ends; the use of phosphorothioate or 2′ 0-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

ii. Vectors

In other related aspects, the invention includes an isolated nucleic acid encoding an inhibitor, wherein the inhibitor such as an siRNA, inhibits tRNA expression, function, or activity, or a regulator thereof, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). In another aspect of the invention, tRNA, or a regulator thereof, can be inhibited by way of inactivating and/or sequestering tRNA, or a regulator thereof. As such, inhibiting the effects of tRNA can be accomplished by using a transdominant negative mutant.

In another aspect, the invention includes a vector comprising an siRNA polynucleotide. Preferably, the siRNA polynucleotide is capable of inhibiting the expression of a target, wherein the target is selected from the group consisting of tRNA, or regulators thereof. The incorporation of a desired polynucleotide into a vector and the choice of vectors is well-known in the art as described in, for example, Sambrook et al., supra, and Ausubel et al., supra.

The siRNA polynucleotide can be cloned into a number of types of vectors. However, the present invention should not be construed to be limited to any particular vector. Instead, the present invention should be construed to encompass a wide plethora of vectors which are readily available and/or well-known in the art. For example, an siRNA polynucleotide of the invention can be cloned into a vector including, but not limited to a plasmid, a phagemid, a phage derivative, an animal viruses, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the siRNA, afleast one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosih promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

d. Peptides

When the tRNA inhibitor is a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the a-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the a-amino protecting group, and the FMOC method, which utilizes 9-fluorenylmethyloxcarbonyl to protect the a-amino of the amino acid residues. Both methods are well-known by those of skill in the art. Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a tRNA inhibitor in accordance with the invention, a peptide is purified to remove contaminants. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

The present invention also includes tRNA inhibitors comprising proteins, such as enzymes, especially RNase. One such example is Onconase, which is identified for the first time, herein, as an RNase specific for tRNA.

e. Small Molecules

When the tRNA inhibitor is a small molecule, a small molecule activator may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

2. Activators of tRNA Expression, Function, or Activity

a. Peptides

When the tRNA activator is a peptide, the peptide may be chemically synthesized or modified as described elsewhere herein.

b. Nucleic Acids

When the tRNA activator comprises a nucleic acid, any number of procedures may be used for the generation of an isolated nucleic acid encoding the agonist as well as derivative or variant forms of the isolated nucleic acid, using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) and by direct synthesis. For recombinant and in vitro transcription, DNA encoding RNA molecules can be obtained from known clones, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al.

An isolated nucleic acid of the present invention can be produced using conventional nucleic acid synthesis or by recombinant nucleic acid methods known in the art and described elsewhere herein (Sambrook et al., 2001, Molecular Cloning: A Laboratory Manual, Cold Spring Harbor Laboratory Press, New York) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes.

In the phosphotriester method, oligonucleotides are prepared which can then be joined together to form longer nucleic acids. For a description of this method, see Narang et al., (1979, Meth. Enzymol., 68: 90) and U.S. Pat. No. 4,356,270. The phosphotriester method can be used in the present invention to synthesize an isolated tRNA activator nucleic acid. In addition, the compositions of the present invention can be synthesized in whole or in part, or an isolated tRNA activator nucleic acid can be conjugated to another nucleic acid using organic synthesis such as the phosphodiester method, which has been used to prepare a tRNA gene. See Brown et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing nucleic acids, described in U.S. Pat. No. 4,293,652, is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention. tRNA activators may include small synthetic nucleic acid compounds. Thus, oligonucleotide agents are incorporated herein and include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (1994, Nucleic Acids Res. 22: 2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body.

As nucleic acids are polymers of subunits or monomers, many of the modifications described below occur at a position which is repeated within a nucleic acid, e.g., a modification of a base, or a phosphate moiety, or a non-linking 0 of a phosphate moiety. In some cases the modification will occur at all of the subject positions in the nucleic acid but in many, and in fact in most cases it will not. By way of example, a modification may only occur at a 3′ or 5′ terminal position, in a terminal region, e.g., at a position on a terminal nucleotide, or in the last 2, 3, 4, 5, or 10 nucleotides of a strand. A component can be attached at the 3′ end, the 5′ end, or at an internal position, or at a combination of these positions. For example, the component can be at the 3′ end and the 5′ end; at the 3′ end and at one or more internal positions; at the 5′ end and at one or more internal positions; or at the 3′ end, the 5′ end, and at one or more internal positions. For example, a phosphorothioate modification at a non-linking 0 position may only occur at one or both termini, or may only occur in a terminal region, e.g., at a position on a terminal nucleotide or in the last 2, 3, 4, 5, or 10 nucleotides of the oligonucleotide. The 5′ end can be phosphorylated.

For increased nuclease resistance and/or binding affinity to the target, an oligonucleotide agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, 0-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino) It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

Preferred substitutents include but are not limited to 2′-methoxyethyl, 2′-OCH3, 2′-O-allyl, 2′-C-allyl, and 2′-fluoro.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

One way to increase resistance is to identify cleavage sites and modify such sites to inhibit cleavage. For example, the dinucleotides 5′-UA-3′, 5′-UG-3′, 5′-CA-3′, 5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. In certain embodiments, all the pyrimidines of the miRNA inhibitor carry a 2′-modification, and the miRNA inhibitor therefore has enhanced resistance to endonucleases.

In addition, to increase nuclease resistance, the 2′ modifications can be used in combination with one or more phosphate linker modifications (e.g., phosphorothioate). The so-called “chimeric” oligonucleotides are those that contain two or more different modifications. With respect to phosphorothioate linkages that serve to increase protection against RNase activity, the miRNA inhibitor can include a phosphorothioate at least the first, second, or third internucleotide linkage at the 5′ or 3′ end of the nucleotide sequence. In one embodiment, the miRNA inhibitor includes a 2′-modified nucleotide, e.g., a 2′-deoxy, 2′-deoxy-2′-fluoro, 2′-0-methyl, 2′-O-methoxyethyl (2′-O-MOE), 21-0-aminopropyl (2′-O-AP), 2¹-0-dimethylaminoethyl (2′-O-DMAOE), 2′-0-dimethylaminopropyl (2′-O-DMAP), 2′-O-dimethylaminoethyloxyethyl (2′-O-DMAEOE), or 2′-O—N-methylacetamido (2′-O-NMA). In a preferred embodiment, the miRNA inhibitor includes at least one 2′-O-methyl-modified nucleotide, and in some embodiments, all of the nucleotides of the miRNA inhibitor include a 2′-O-methyl modification.

The 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

The oligonucleotide can be constructed using chemical synthesis and/or enzymatic ligation reactions using procedures known in the art. For example, an oligonucleotide can be chemically synthesized using naturally occurring nucleotides or variously modified nucleotides designed to increase the biological stability of the molecules or to increase the physical stability of the duplex formed between the oligonucleotide and target nucleic acids, e.g., phosphorothioate derivatives and acridine substituted nucleotides can be used. Other appropriate nucleic acid modifications are described herein. Alternatively, the oligonucleotide can be produced biologically using an expression vector into which a nucleic acid has been subcloned in an antisense orientation (i.e., RNA transcribed from the inserted nucleic acid will be of an antisense orientation to a target nucleic acid of interest (e.g., an mRNA, pre-mRNA, or an miRNA).

Any polynucleotide of the invention may be further modified to increase its stability in vivo. Possible modifications include, but are not limited to, the addition of lanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

c. Small Molecules

When the tRNA activator is a small molecule, a small molecule activator may be obtained using standard methods known to the skilled artisan, and described elsewhere herein.

### Methods:

### 1. Methods of Regulating Cell Apoptosis

The present invention provides a method of inhibiting cell apoptosis in a mammal, thereby increasing or promoting cell survival. The method of the invention comprises administering a therapeutically effective amount of at least one tRNA activator to a mammal wherein a tRNA activator attenuates, or halts the pathophysiological changes associated with the intrinsic apoptosis pathway in a cell, including cell death. In one embodiment the cell is a mammalian cell. In another embodiment the cell is a human cell.

The present invention provides a method of augmenting cell apoptosis in a mammal, thereby decreasing cell survival. The method of the invention comprises administering a therapeutically effective amount of at least one tRNA inhibitor to a mammal wherein a tRNA inhibitor augments the pathophysiological changes associated with the intrinsic apoptosis pathway in a cell, including cell death. In one embodiment the cell is a mammalian cell. In another embodiment the cell is a human cell. In still another embodiment the cell is a cancer cell.

The present invention further includes a method of increasing tRNA expression function or activity in a cell, said method comprising contacting said cell with a tRNA activator, wherein when said tRNA activator contacts said cell, said tRNA activator augments said tRNA expression, function, or activity in said cell. In one embodiment the tRNA activator is selected from the group consisting of a protein, a peptide, an siRNA, a ribozyme, an antisense, an aptamer, a peptidomimetic, a small molecule, or any combination thereof. In another embodiment the cell is a mammalian cell. In another embodiment the cell is a human cell. In still another embodiment the cell is a cancer cell.

The present invention further includes a method of inhibiting tRNA expression function or activity in a cell, said method comprising contacting said cell with a tRNA inhibitor, wherein when said tRNA inhibitor contacts said cell, said tRNA inhibitor inhibits said tRNA expression, function, or activity in said cell. In one embodiment the tRNA inhibitor is selected from the group consisting of a protein, a peptide, an siRNA, a ribozyme, an antisense, an aptamer, a peptidomimetic, a small molecule, or any combination thereof. In another embodiment the cell is a mammalian cell. In another embodiment the cell is a human cell.

The present invention also includes a method for inhibiting an interaction between cytochrome c and Apaf-1 in a cell, said method comprising contacting said cell with an effective amount of a tRNA activator, wherein said tRNA activator increases tRNA expression, activity, stability, or function in said cell, thereby inhibiting said interaction between cytochrome c and Apaf-1. In one embodiment the tRNA activator is selected from the group consisting of a protein, a peptide, an siRNA, a ribozyme, an antisense, an aptamer, a peptidomimetic, a small molecule, or any combination thereof. In another embodiment the cell is a mammalian cell. In another embodiment the cell is a human cell. In still another embodiment the cell is a cancer cell.

The present invention also includes a method for increasing an interaction between cytochrome c and Apaf-1 in a cell, said method comprising contacting said cell with an effective amount of a tRNA inhibitor, wherein said tRNA inhibitor decreases tRNA expression, activity, stability, or function in said cell, thereby increasing said interaction between cytochrome c and Apaf-1. In one embodiment the tRNA inhibitor is selected from the group consisting of a protein, a peptide, an siRNA, a ribozyme, an antisense, an aptamer, a peptidomimetic, a small molecule, or any combination thereof. In another embodiment the cell is a mammalian cell. In another embodiment the cell is a human cell.

The subject may be diagnosed with a disease or disorder wherein the disease or disorder has a dysregulation of the intrinsic apoptosis pathway in a cell as part of the disease's clinical features. Examples of a disease or disorder which may be treated using the methods of the present invention include but are not limited to cancer. In a preferred embodiment the subject is a mammal. In a more preferred embodiment the subject is a human.

A tRNA regulator of the instant invention may be used either alone or in combination with other therapeutic agents to treat a subject. A tRNA regulator may be administered either, before, during, after, or throughout the administration of said therapeutic agent. The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like. In a preferred embodiment, a tRNA, tRNA-like molecule, or a tRNA regulator is administered to a subject in need thereof in combination with a chemotherapeutic agent. In one embodiment the chemotherapeutic agent is doxorubicin. The chemotherapeutic agent may be administered before, concurrently with, or after administration of the tRNA, tRNA-like molecule, or tRNA regulator.

Based on the disclosure provided herein, a skilled artisan would understand that the compositions of the invention can be used to treat a disease associated with abnormal cyctochrome c release. Given that cyctochrome c release is associated with apoptosis, the present invention provides methods useful for preventing or treating a wide variety of diseases and pathological conditions where inappropriate apoptosis is involved or has a causal role in the pathophysiology, and is characterized by aberrant levels of apoptotic activity in a cell or tissue. These diseases or conditions in which enhanced apoptosis contributes to the underlying pathogenesis and which are referred to as apoptosis-related diseases and conditions in the specification and claims, include, but are not limited to, neurodegenerative diseases, autoimmune and inflammatory disorders, infectious diseases (including viral hepatitis), inflammatory bowel disease, ischemia/hypoperfusion, and sepsis amongst others.

Neurodegenerative diseases may consist of, but are not limited to, Parkinson's Disease, including early onset forms (Autosomal recessive juvenile Parkinson's; ARJP), Lewy body dementias, and general synucleinopathies; Alzheimer's disease, including frontotemporal dementias (FTD), corticobasal degeneration (CBD), progressive supranuclear palsy (PSP), and general tauopathies and amyloidopathies; Amyotrophic Lateral Sclerosis, including adult-onset motor neuron disease; and Huntington's disease, including spino-cerebellar ataxias and adult onset trinucleotide repeat disorders.

Autoimmune and inflammatory disorders may include, but are not limited to, arthritic diseases such as rheumatoid arthritis, osteoarthritis, gouty arthritis, spondylitis; Behcet disease; sepsis, septic shock, endotoxic shock, gram negative sepsis, gram positive sepsis, and toxic shock syndrome; multiple organ injury syndrome secondary to septicemia, trauma, or hemorrhage; ophthalmic disorders such as allergic conjunctivitis, vernal conjunctivitis, uveitis, and thyroid-associated ophthalmopathy; eosinophilic granuloma; pulmonary or respiratory disorders such as asthma, chronic bronchitis, allergic rhinitis, ARDS, chronic pulmonary inflammatory disease (e.g., chronic obstructive pulmonary disease), silicosis, pulmonary sarcoidosis, pleurisy, alveolitis, vasculitis, pneumonia, bronchiectasis, and pulmonary oxygen toxicity; reperfusion injury of the myocardium, brain, or extremities; fibrosis such as cystic fibrosis; keloid formation or scar tissue formation; atherosclerosis; autoimmune diseases such as systemic lupus erythematosus (SLE), autoimmune thyroiditis, multiple sclerosis, some forms of diabetes, and Reynaud's syndrome; connective tissue disease, autoimmune pulmonary inflammation, Guillain Barre syndrome, autoimmune thyroiditis, insulin dependent diabetes mellitis, myasthenia gravis, graft versus host disease and autoimmune inflammatory eye disease; transplant rejection disorders such as GVHD and allograft rejection, chronic glomerulonephritis; inflammatory bowel diseases such as Crohn's disease, ulcerative colitis and necrotizing enterocolitis, inflammatory dermatoses such as contact dermatitis, atopic dermatitis, psoriasis, or urticaria, fever and myalgias due to infection; central or peripheral nervous system inflammatory disorders such as meningitis, encephalitis, and brain or spinal cord injury due to minor trauma; Sjorgren's syndrome; diseases involving leukocyte diapedesis; alcoholic hepatitis; bacterial pneumonia; antigen-antibody complex mediated diseases; hypovolemic shock; Type I diabetes mellitus; acute and delayed hypersensitivity; disease states due to leukocyte dyscrasia and metastasis; thermal injury; granulocyte transfusion associated syndromes; cytokine-induced toxicity; and allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiform, Stevens Johnson syndrome, allergic conjunctivitis, atopic keratoconjunctivitis, venereal keratoconjunctivitis, giant papillary conjunctivitis and contact allergies), such as asthma (particularly allergic asthma) or other respiratory problems.

Infectious diseases amenable to prevention or treatment according to the invention include, but are not limited to, anthrax, bovine spongiform encephalopathy (BSE), chicken pox, cholera, cold, conjunctivitis, Creutzfeldt Jakob Disease (CJD), Dengue fever, diphtheria, ebola, viral encephalitis, Fifth's disease, hand, foot, and mouth disease (HFMD), Hantavirus, Helicobacter Pylori, hepatitis, herpes, hookworm, influenza, Lassa fever, Lyme disease, Marburg hemorrhagic fever, measles, meningitis, mononucleosis, mucormycosis, mumps, nosocomial infections, otitis media, pelvic inflammatory disease (PID), plague, pneumonia, polio, prion diseases, rabies, rheumatic fever, Rocky Mountain spotted fever, roseola, Ross River virus infection, rubella, scarlet fever, sexually transmitted diseases (STDs), shingles, smallpox, Strep throat, tetanus, toxic shock syndrome (TSS), toxoplasmosis, trachoma, tuberculosis, tularemia, typhoid fever, whooping cough, and yellow fever

Hepatitis, an inflammation of the liver cause by a range of factors including toxins, ischemia, drugs and one of several hepatitis viruses, is another disease amenable to prevention or treatment according to the invention. There are several types of hepatitis virus infections, including hepatitis A, B, and C. Hepatitis A is considered the least threatening since it generally does not lead to liver damage, and 99% of those infected fully recover. Hepatitis B is a serious viral disease that attacks the liver. Approximately 2-10% of adults and 25-80% of children under the age of 5 will not be able to clear the virus in six months and are considered to be chronically infected. Hepatitis C also causes inflammation of the liver, with an estimated 80% of those infected developing chronic hepatitis. Many can develop cirrhosis (scarring of the liver), and some may also develop liver cancer. All forms of hepatitis (both viral and nonviral causes) are amenable to prevention or treatment with the present invention.

Ischemic conditions amenable to prevention or treatment according to the invention include, but are not limited to, cardiac, neural, mesenchymal, and limb ischemia. Ischemia, as set out above, is a condition resulting from insufficient supply of blood, usually caused by arterial blockage, to a tissue or organ. Myocardial infarction and stroke are some of the ischemic conditions amenable to prevention or treatment of the invention.

“Sepsis” is the term used to describe systemic inflammation caused by an overwhelming bacterial infection, pancreatitis, ischemia, toxins etc. Sepsis is a diffuse inflammatory state induced by a variety of potential stimuli including bacterial infection and can also be referred to as systemic inflammatory response syndrome (SIRS). The inflammatory state which ensues is associated with lymphopenia, impaired tissue perfusion, and ultimately end organ dysfunction including brain, lungs, heart, liver, kidney, and muscle. Uncomplicated sepsis, such as that caused by flu and other viral infections, gastroenteritis, or dental abscesses, is very common Severe sepsis, arises when sepsis occurs in combination with problems in one or more of the vital organs, such as the heart, kidneys, lungs, or liver. Septic shock occurs when sepsis is complicated by low blood pressure that does not respond to standard treatment (fluid administration) and leads to problems in one or more of the vital organs as set out above. This condition has a high mortality rate (around 50%) and is characterized by a lack of oxygen to vital cells, tissues, and organs and extreme reduction in blood pressure.

The invention also concerns methods for treating or preventing cancer in a patient, wherein the method comprises administering to the patient an effective amount of the composition of the present invention. The subject method can be used to treat or prevent cancers including, but not limited to, lung cancer, breast cancer, colon cancer, prostate cancer, melanomas, pancreatic cancer, stomach cancer, liver cancer, brain cancer, kidney cancer, uterine cancer, cervical cancer, ovarian cancer, cancer of the urinary tract, gastrointestinal cancer, head-and-neck cancer, or leukemia.

The diseases and conditions preventable or treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

As a function of regulating apoptosis in a cell, the invention provides compositions and methods for treating a disease associated with abnormal cyctochrome c release. In some instances, abnormal cyctochrome release is associated with increased apoptosis. Non-limiting examples of diseases associated with increased apoptisis includes but are not limited to AIDS; neurodegenerative disorders, in particular Alzheimer's disease, Parkinson's disease, amyotrophic lateral sclerosis, retinitis pigmentosa, spinal muscular atrophy, cerebellar degeneration; myelodysplastic syndromes, in particular aplastic anemia; ischemic injury, in particular myocardial infarction, stroke, reperfusion injury; toxin-induced liver disease through alcohol abuse, or abuse of other substances; diseases with an inappropriate level of production or secretion of hormones, in particular hyperthyroidismus; diseases characterized by inappropriate bone metabolism; metabolic diseases; degenerative processes associated with injury or surgery; and degenerative processes due the hormonal cycle in females, including women.

Recent advances have implicated enhanced apoptosis in the pathogenesis of a broad cross section of human diseases or physiological insults including various forms of neurodegenerative diseases, autoimmune and inflammatory disorders, infectious diseases (such as from bacteria, viruses, protozoa, hepatitis, inflammatory bowel disease, etc.), ischemia/hypoperfusion, sepsis, ionizing and UV irradiation, chemotherapeutic agents and toxins, induce apoptosis in affected organs and tissues.

In another embodiment, the invention provides compositions and methods for treating a disease associated with abnormal cyctochrome c release wherein the abnormal cyctochrome c release results in decreased apoptosis. Non-limiting examples of diseases associated with decreased apoptisis includes but are not limited to malignant and benign hyperproliferative diseases, in particular lymphomas, carcinomas, sarcomas, other tumors, or leukemias; autoimmune disorders, in particular systemic lupus erythematosus, rheumatoid arthritis, psoriasis, inflammatory bowel disease, or autoimmune diabetes mellitus; and viral infections, in particular those of retroviruses, herpesviruses, poxviruses or adenoviruses.

2. Methods of Delivering a tRNA Regulator to a Cell

The present invention comprises a method for regulating cell survival in a mammal, said method comprising administering a therapeutic amount of a tRNA regulator to said mammal. In particular, the invention includes a method for attenuating cell apoptosis. In another embodiment, the invention includes a method for enhancing cell apoptosis. Isolated tRNA regulators can be delivered to a cell in vitro or in vivo using viral vectors comprising one or more isolated tRNA regulator sequences.

Generally, the nucleic acid sequence has been incorporated into the genome of the viral vector. The viral vector comprising an isolated regulator nucleic acid described herein can be contacted with a cell in vitro or in vivo and infection can occur. The cell can then be used experimentally to study, for example, the effect of an isolated tRNA regulator in vitro, or the cells can be implanted into a subject for therapeutic use. The cell can be migratory, such as a hematopoietic cell, or non-migratory. The cell can be present in a biological sample obtained from the subject (e.g., blood, bone marrow, tissue, fluids, organs, etc.) and used in the treatment of disease, or can be obtained from cell culture.

After contact with the viral vector comprising an isolated tRNA regulator nucleic acid sequence, the sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is removed from the subject or animal and returned to the subject or animal once contacted with the viral vector comprising the isolated nucleic acid of the present invention. Ex vivo gene therapy has been described, for example, in Kasid et al., Proc. Natl. Acad. Sci. USA 87:473 (1990); Rosenberg et al, New Engl. J Med. 323:570 (1990); Williams et al., Nature 310476 (1984); Dick et al., Cell 42:71 (1985); Keller et al., Nature 318:149 (1985) and Anderson et al., U.S. Pat. No. 5,399,346 (1994).

Where a cell is contacted in vitro, the cell incorporating the viral vector comprising an isolated nucleic acid can be implanted into a subject or experimental animal model for delivery or used in in vitro experimentation to study cellular events mediated by tRNA regulation of the apoptosis pathway.

Various viral vectors can be used to introduce an isolated nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated nucleic acid of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, an isolated nucleic acid can be delivered to cells without vectors, e.g. as “naked” nucleic acid delivery using methods known to those of skill in the art. See, for example, U.S. Pat. Nos. 5,350,674 and 5,585,362.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a polynucleotide into a host cell include colloidal dispersion systems, such as macromolecule complexes, nanocapsules, microspheres, beads, and lipid-based systems including oil-in-water emulsions micelles mixed micelles, and liposomes. A preferred colloidal system for use as a delivery vehicle in vitro and in vivo is a liposome (i.e., an artificial membrane vesicle). The preparation and use of such systems is well known in the art.

Various forms of an isolated nucleic acid, as described herein, can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated nucleic acid of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of polynucleotides into a cell. Further, liposomes can be used as carriers to deliver a nucleic acid to a cell, tissue or organ. Liposomes comprising neutral or anionic lipids do not generally fuse with the target cell surface, but are taken up phagocytically, and the polynucleotides are subsequently subjected to the degradative enzymes of the lysosomal compartment (Straubinger et al., 1983, Methods Enzymol. 101:512-527; Mannino et al., 1988, Biotechniques 6:682-690).. However, as demonstrated by the data disclosed herein, an isolated snRNA of the present invention is a stable nucleic acid, and thus, may not be susceptible to degradative enzymes. Further, despite the fact that the aqueous space of typical liposomes may be too small to accommodate large macromolecules, the isolated nucleic acid of the present invention is relatively small, and therefore, liposomes are a suitable delivery vehicle for the present invention. Methods of delivering a nucleic acid to a cell, tissue or organism, including liposome-mediated delivery, are known in the art and are described in, for example, Felgner (Gene Transfer and Expression Protocols Vol. 7, Murray, E. J. Ed., Humana Press, New Jersey, (1991)).

In other related aspects, the invention includes an isolated nucleic acid operably linked to a nucleic acid comprising a promoter/regulatory sequence such that the nucleic acid is preferably capable of delivering an isolated nucleic acid. Thus, the invention encompasses expression vectors and methods for the introduction of an isolated nucleic acid into or to cells.

Such delivery can be accomplished by generating a plasmid, viral, or other type of vector comprising an isolated nucleic acid operably linked to a promoter/regulatory sequence which serves to introduce the tRNA regulator into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated nucleic acid may be accomplished by placing an isolated nucleic acid, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

### 3. Pharmaceutical Compositions and Therapies

Administration of a tRNA activator or tRNA inhibitor comprising one or more peptides, small molecules, or nucleic acids of the invention in a method of treatment can be achieved in a number of different ways, using methods known in the art.

The therapeutic methods of the invention thus encompass the use of pharmaceutical compositions comprising a tRNA activator or tRNA inhibitor peptide, fusion protein, small molecule, of the invention and/or an isolated nucleic acid to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 uM and 10 uM in a mammal

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 pg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts.

Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### 4. Methods of Identifying Potential Therapeutic Agents

The disclosure presented herein demonstrates that RNA (e.g., tRNA) inhibits the interaction of cytochrome c with Apaf-1. By binding to cytochrome c and preventing its association with Apaf-1, tRNA can directly regulate apoptosis. Accordingly, the invention provides a method to screen for agents that disrupt or promote binding between tRNA and cytochrome c.

Without wishing to be bound by any particular theory, it is believed that tRNA binding to cytochrome c prevents cytochrome c to associate with with Apaf-1, thereby promoting cell survival. Thus, blocking or disrupting the interaction between cytochrome c and tRNA prevents tRNA from inhibiting cyctochrome c interaction with Apaf-1, thereby promoting apoptosis. Alternatively, promoting the interaction between cytochrome c and tRNA induces tRNA to promote cell survival because the apoptotic promoting interaction between cytochrome c and Apaf-1 is inhibited. Accordingly, any screening method in the art to identify an agent that modulates the formation of an RNA-protein complex (e.g., tRNA-cyctochrome c) formed in vivo or in vitro can be used to identify the desired agent (e.g., inhibitor or activator).

In one embodiment, the invention provides a method of screening for an agent that modulates or regulates the formation of a tRNA-cyctochrome c complex formed in vivo or in vitro. In one embodiment, the screening method comprises contacting a tRNA-cyctochrome c complex with a test agent under conditions that are effective for tRNA-cyctochrome c complex formation and detecting whether or not the test agent disrupts tRNA-cyctochrome c, wherein detection of disruption of tRNA-cyctochrome c interaction identifies an agent that disrupts tRNA-cyctochrome c interaction.

Other methods, as well as variation of the methods disclosed herein will be apparent from the description of this invention. For example, the test compound may be either fixed or increased, a plurality of compounds or proteins may be tested at a single time. “Modulation”, “modulates”, and “modulating” can refer to enhanced formation of the tRNA-cyctochrome c complex, a decrease in formation of the tRNA-cyctochrome c complex, a change in the type or kind of the tRNA-cyctochrome c complex or a complete inhibition of formation of the tRNA-cyctochrome c complex. Suitable compounds that may be used include but are not limited to proteins, nucleic acids, small molecules, hormones, antibodies, peptides, antigens, cytolines, growth factors, pharmacological agents including chemotherapeutics, carcinogenics, or other cells (i.e. cell-cell contacts). Screening assays can also be used to map binding sites on RNA or protein. For example, tag sequences encoding for RNA tags can be mutated (deletions, substitutions, additions) and then used in screening assays to determine the consequences of the mutations.

The invention relates to a method for screening test agents, test compounds or proteins for their ability to modulate or regulate tRNA-cyctochrome c complex. By performing the methods of the present invention for purifying tRNA-cyctochrome c complexes formed in vitro or in vivo and observing a difference, if any, between the tRNA-cyctochrome c complexes purified in the presence and absence of the test, agents, test compounds or proteins, wherein a difference indicates that the test agents, test compounds or proteins modulate the tRNA-cyctochrome c complex.

One aspect of the invention is a method for detecting an agent that is capable of interfering with the interaction between tRNA-cyctochrome c. An agent that interferes with tRNA-cyctochrome c complex would be expected to promote apoptosis. Alternatively, an agent that promotes formation of tRNA-cyctochrome c complex would be expected to promote cell survival.

In one embodiment, the method comprises: (a) contacting an agent with a mixture comprising tRNA and cyctochrome c under conditions that are effective for tRNA-cyctochrome c complex formation; and (b) detecting whether the presence of the agent decreases or increases the level of tRNA-cyctochrome c complex formation. In some instances, the agent binds to tRNA and thereby inhibits tRNA-cyctochrome c complex complex formation. In another instance, the agent binds to cyctochrome c and thereby inhibits tRNA-cyctochrome c complex formation. Any of a variety of conventional procedures can be used to carry out such an assay.

In another embodiment, the method comprises: (a) contacting an agent with a mixture comprising tRNA-cyctochrome c complex under conditions that are effective for maintaining tRNA-cyctochrome c complex; and (b) detecting whether the presence of the agent disrupts the A3G:RNA complex. In some instances, the agent binds to cytochrome c and thereby disrupts tRNA-cyctochrome c complex. In another instance, the agent binds to tRNA and thereby disrupts tRNA-cyctochrome c complex complex formation. Any of a variety of conventional procedures can be used to carry out such an assay.

The skilled artisan would also appreciate, in view of the disclosure provided herein, that standard binding assays known in the art, or those to be developed in the future, can be used to assess the binding of tRNA and cyctochrome c in the presence or absence of the test compound to identify a useful compound. Thus, the invention includes any compound identified using this method.

In one embodiment, the invention provides methods for identifying associative interactions between a target molecule and a candidate agent, such as an associative interaction of a therapeutic agent and a biomolecule, which results in formation of a molecular complex. Associative interactions in the present invention includes the association of a single candidate agent and a single target molecule, and also includes association of a plurality of candidate agents and one or more target molecules. In addition, the present methods are useful for identifying non-associative interactions between a target molecule and a candidate agent that result in a change in the composition and/or structure of the candidate agent, target molecule or both, such as post-translational or co-translational processes, enzymatic reactions or molecular complex formation reactions involving one or more proteins.

In one embodiment, a molecular complex is contacted with chemistries from a library of compounds. In such instances, the molecular complex is exposed to a library of compounds with the intent of identify compounds that disrupt the molecular complex.

In one embodiment, components of a molecular complex is contacted with chemistries from a library of compounds. In such instances, the components of a molecule complex are exposed to a library of compounds with the intent of identify those compounds that prevent complex formation. In this context, the invention provides a method of identifying compounds that block the interaction between components of a molecular complex.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Reagents, Plasmids, and Protein Preparation

The following reagents were obtained from the indicated sources: Bovine cytochrome c, rRNA, tRNA, doxorubicin, proteinase K, formaldehyde, RNase A, empigen, zVADFMK, and anti-Flag M2 beads (Sigma); Protein A agarose and Texas Red-labeled dextran (Invitrogen); RNase Inhibitor (Promega); antibodies against caspase-9 (MBL International Corporation), caspase-3 (Santa Cruz Biotechnology), Smac (Cell Signaling), and actin (Sigma). Anti-cytochrome c antibodies for immunoblotting and immunoprecipitation were purchased from R&D systems and BD Pharmingen, respectively. Anti-Apaf-1 antibody was kindly provided by Dr. X. Wang (Zou et al., 1997). Onconase was kindly provided by the Alfacell Corporation (Somerset, N.J.). Flagcaspase-9-pRKS was previously described (Chang et al., 2003). Recombinant full-length Apaf-1 (amino acids 1-1248) was expressed in Hi-5 insect cells and purified as described (Bao et al., 2007; Jiang and Wang, 2000). Apaf-1 (1-591) was expressed in BL21 (DE3) Escherichia coli strain and purified as described (Riedl et al., 2005).

tRNA Sequences
Human Mitochondrial tRNA

Ala:
SEQ ID NO. 1
AAGGGCTTAGCTTAATTAAAGTGGCTGATTTGCGTTCAGTTGATGCAG
AGTGGGGTTTTGCAGTCCTTA
Leu:
SEQ ID NO. 2
ACTTTTAAAGGATAACAGCTATCCATTGGTCTTAGGCCCCAAAAATTT
TGGTGCAACTCCAAATAAAAGTA
Met:
SEQ ID NO. 3
AGTAAGGTCAGCTAAATAAGCTATCGGGCCCATACCCCGAAAATGTTG
GTTATACCCTTCCCGTACTA
Phe:
SEQ ID NO. 4
GTTTATGTAGCTTACCTCCTCAAAGCAATACACTGAAAATGTTTAGAC
GGGCTCACATCACCCCATAAAC A
Ser:
SEQ ID NO. 5
GAGAAAGCTCACAAGAACTGCTAACTCATGCCCCCATGTCTAACAACA
TGGCTTTCTCA

Human tRNAs (cytoplasmic)

Gln(CTG):
SEQ ID NO. 6
GGTTCCATGGTGTAATGGTAAGCACTCTGGACTCTGAATCCAGCCATC
TGAGTTCGAGTCTCTGTGGAACCT
Ser:
SEQ ID NO. 7
GTAGTCGTGGCCGAGTGGTTAAGGCGATGGACTAGAAATCCATTGGGG
TCTCCCCGCGCAGGTTCGAATCCTGCCGACTACG
Trp:
SEQ ID NO. 8
GACCTCGTGGCGCAACGGTAGCGCGTCTGACTCCAGATCAGAAGGTTG
CGTGTTCAAATCACGTCGGGGTCA
Asp:
SEQ ID NO. 9
TCCTTGTTACTATAGTGGTGAGTATCTCTGCCTGTCATGCGTGAGAGA
GGGGGTCGATTCCCCGACGGGGAG

### Caspase Activation Assay and Apaf-1:Cytochrome-c Binding Assay

S 100 cell extracts of Jurkat and HeLa cells were prepared as described (Liu et al., 1996). Activation of caspase-9 and caspase-3 in the S 100 extracts was induced by the addition of 20 pg/ml cytochrome c and subsequent incubation at 37° C. for 1 h. Recombinant procaspase-9 was produced by a coupled in vitro-transcription and translation system (Promega) in the presence of ³⁵S-methionine. Activation of in vitro-translated, ³⁵S-labeled caspase-9 was induced by incubation with purified Apaf-1 (10 nM) or Apaf-1 (1-591) (20 nM), cytochrome c (20 pg/ml), and dATP (1 mM) for 1 h at 30° C. in oligomerization buffer (20 mM Hepes/pH 7.4, 10 mM KCl, 1.5 mM MgCl₂, 2 mM EDTA, and 1 mM DTT).

To assess the binding of cytochrome c to Apaf-1 in the presence of RNA, recombinant full-length Apaf-1 immobilized on Ni-NTA beads was incubated without or with cytochrome c, or with cytochrome c plus total cellular RNA at 4° C. for 1 h.

### Gel Filtration Assay

Gel filtration analysis was performed on a Superose 6 HR 10/30 column driven by an Akta FPLC system (GE Healthcare). The column was calibrated with molecular weight standards from Bio-Rad. 500 μg of S100 extracts were injected into the column. The buffer contained 20 mM Hepes (pH 7.0), 0.1% CHAPS, 5 mM DTT, 5% Sucrose, and 50 mM NaCl. 500 μl was collected for each fraction. To analyze interaction of total RNA with cytochrome c, total RNA was incubated with cytochrome c at 25° C. for 15 min.

RNA Preparation and tRNA:Cytochrome c Interaction

Total RNA was prepared using Trizol reagents (Invitrogen) following manufacturer's instructions. mRNA was further purified using Oligo(dT)-Cellulose (GE Healthcare). RNAs were dissolved in TE buffer (10 mM Tris-HCl, pH7.5, 1 mM EDTA), and the concentration was quantified by NanoVue spectrophotometer (GE Healthcare). To assess the effect of RNA on the binding of cytochrome c to Apaf-1, recombinant full-length Apaf-1 immobilized on Ni-NTA beads was incubated without or with cytochrome c, or with cytochrome c plus total cellular RNA at 4° C. for 1 h. To examine the effect of RNA on caspase-9 activation, 2 μl of RNAs were added to a total of 20 μl of Jurkat S100 extracts.

To examine the tRNA:cytochrome c interaction in vivo, HeLa cells grown in DMEM supplemented with 10% FBS were harvested and washed twice with ice-cold PBS. Cell pellets were re-suspended in PBS containing 0.2% formaldehyde and incubated at room temperature for 10 min. The cross-linking reaction was quenched with 0.15 M (final concentration) of glycine, pH 7.4. Cell extracts were prepared in the irruminoprecipitation buffer (20 mM Tris, pH 7.8, 500 mM NaCl, and 2.5 mM MgCl2) containing 1% empigen, and incubated with anti-cytochrome c, anti-Smac/Diablo, or an isotype-matching control antibodies at 4° C. for 1 h, followed by incubation with protein G agarose beads for an additional 2 h Immunoprecipitates were treated with proteinase K and RNA was extracted by phenol/chloroform. Purified RNA was separated by 8% denaturing polyacrylamide gel electrophoresis and analyzed by Northern blotting. Oligonucleotide sequences used to probe various RNAs are as follows:

Mit. tRNA^{Ala}:
SEQ ID NO. 10
5′-TGCAAAACCCCACTCTGCATCAACTGAACGCAAATCAGCCACTTTAATTAAGCTAA
GCCC-3′
Mit. tRNA^{Met}:
SEQ ID NO. 11
5′-TACGGGAAGGGTATAACCAACATTTTCGGGGTATGGGCCCGATAGCTTATTTAGCT
GACC-3′
Mit. tRNA^{Phe}:
SEQ ID NO. 12
5′-GGGGTGATGTGAGCCCGTCTAAACATTTTCAGTGTATTGCTTTGAGGAGGTAAG
CTACAT-3′
Mit. tRNA^{ser}:
SEQ ID NO. 13
5′-AGCTGGTTTCAAGCCAACCCCATGGCCTCC-3′
Cyt. tRNA^{mP}:
SEQ ID NO. 14
5′-CGGGGAATCGACCCCCTCTCTCACGCATGACAGGCAGAGATACTCACCACTA
TAGTAACA-3′
Cyt. tRNA^{Gin}:
SEQ ID NO. 15
5′-TTCAGAGTCCAGAGTGCTTACCATTACACC-3′
Cyt. tRNA^{ser}:
SEQ ID NO. 16
5′-ACCTGCGCGGGGAGACCCCAATGGATTTCTAGTCCATCGCCTTAACCACTCG
GCCACGAC-3′
Cyt. tRNA^{TrP}:
SEQ ID NO. 17
5′-CACCTTCGTGATCATGGTATCTCCC-3′
U1 snRNA:
SEQ ID NO. 18
5′-CACCTTCGTGATCATGGTATCTCCC-3′
5S RNA:
SEQ ID NO. 19
5′-CCTACAGCACCCGGTATTCCCAGGC-3′
RNase MRP:
SEQ ID NO. 20
5′-TGCACGTGGCACTCTCTGCCCGAGG-3′
RNase P:
SEQ ID NO. 21
5′ TCCTGCCCAGTCTGACCTCGCGCGG-3′
7SK:
SEQ ID NO. 22
5′-CGCCTAGCCAGCCAGATCAGCCGAATCAAC-3 ′
7SL:
SEQ ID NO. 23
5′ACCCCTCCTTAGGCAACCTGGTGGTCCCCC-3′
Hvg3:
SEQ ID NO. 24
5′-AGAGGTGGTTTGATGACACG-3′
hY1:
SEQ ID NO. 25
5′-TGAACAATCAATTGAGATAACTCACTACCT-3′

To analyze the tRNA:cytochrome c interaction in vitro, 20,000 cpm of each tRNA was incubated with three different amounts (0.5, 2.5 and 12.5 μM) of recombinant cytochrome c for 45 min at 30° C. in buffer containing 20 mM HEPES, pH 7.5, 20 mM KCl, 2.5 mM MgCl₂, 0.5 mM EDTA, 1 mM DTT, and 0.01% triton X-100 supplemented with salmon sperm DNA (final concentration 50 ng/μl). Sample loading buffer containing urea (final concentration 0.5 M) was added to the reaction mixture and further incubated 10 min at room temperature. The assembled tRNA:cytochrome c mixture was analyzed by 6% native polyacrylamide (acrylamide:bis=79:1) gel electorphoresis followed by autoradiography.

### Microinjection

Microinjection was performed on an Eppendorf micromanipulator/microinjector. HEK293 cells were injected with dextran-Texas Red alone (0.3%, in PBS), plus 0.5 μg/μl cytochrome c, or plus 0.5 μg/μl cytochrome c and 7.5 μg/μl tRNA. 2 h after injection, cells were fixed with 4% paraformaldehyde and mounted with a DAPI-containing medium (Vector Laboratories). For each condition, one hundred to one hundred fifty injected cells were counted, and apoptotic cells were determined by the presence of membrane blebbing and nuclear fragmentation. Data are presented as the means and standard deviations of three independent experiments.

Treatment with Onconase and Doxorubicin

HeLa cells were transfected with onconase using lipofectamine 2000 (Invitrogen) similar to that previously described (Iordanov et al., 2000). 3 h after transfection, the cells were incubated with or without Doxorubicin (1 μg/ml) for another 12 h. Apoptosis was determined by trypan blue exclusion method.

The results of the experiments presented in this Example are now described.

### Example 1

### RNA Hydrolysis Enhances Cytochrome c-Induced Caspase-9 Activation

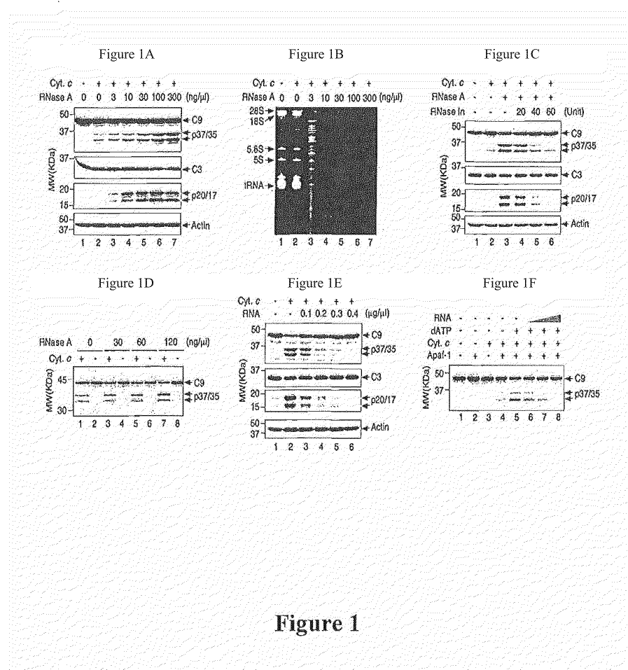

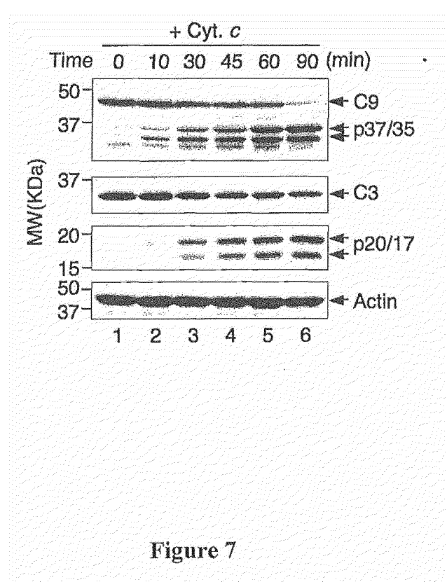

Cytochrome c-induced caspase-9 activation is regulated by nucleotides (Chandra et al., 2006; Kim et al., 2005; Liu et al., 1996; Riedl et al., 2005). The effect of RNA hydrolysis on caspase-9 activation was tested in cell extracts. Addition of cytochrome c to S100 extracts from cell lines such as HeLa and Jurkat results in auto-activation of procaspase-9, generating mature p37/p35 and p10 subunits. This is followed by the processing of the effector procaspase-3 to the mature p20/p17 and p12 subunits (Li et al., 1997) (FIG. 1A and FIG. 7). When HeLa S100 extracts were pretreated with increasing amounts of RNase A, cytochrome c-induced caspase-9 activation was progressively enhanced (FIG. 1A). This stimulatory effect of RNase A correlated with the degradation of cellular RNA in the S100 extracts (FIG. 1B) and was abolished by an RNase inhibitor (FIG. 1C), confirming that enhanced caspase-9 activation was due to the catalytic activity of RNase A. In an analogous system, where cytochrome c was added to reticulocyte lysates containing in vitro-translated procaspase-9 (Liu et al., 2005), treatment with RNase A also enhanced cytochrome c-induced caspase-9 activation (FIG. 1D). When exogenous cellular RNA was added to Jurkat S100 extracts, it inhibited cytochrome c-induced caspase activation in a dose-dependent manner (FIG. 1E).

A reconstituted system was used to assess whether cellular RNA directly inhibits caspase-9 activation or indirectly via other cellular factors. When binding to cytochrome c in the presence of dATP, purified full-length Apaf-1 forms the apoptosome and activates caspase-9 (Zou et al., 1999) (FIG. 1F, lane 5). When exogenous total RNA was included in this system, it strongly inhibited Apaf-1-induced caspase-9 processing at low doses and completely blocked caspase-9 processing at a higher dose (lanes 6-8). This result suggested that RNA might exert its inhibitory effect directly on cytochrome c, Apaf-1, and/or caspase-9.

### Example 2

### RNA Impairs the Cytochrome c:Apaf-1 Interaction and Prevents the Formation of the Apoptosome

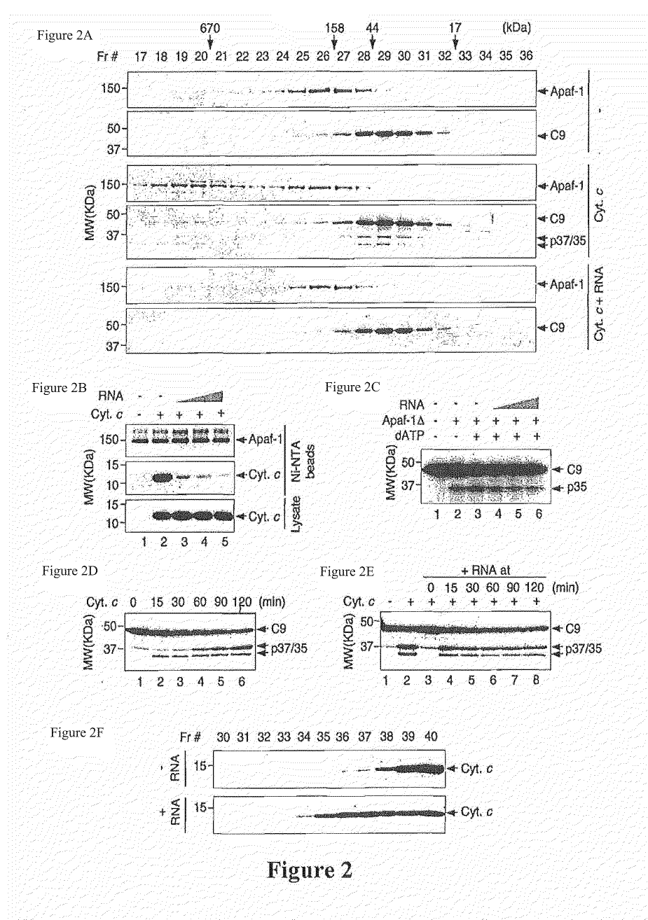

To investigate the mechanism by which cellular RNA inhibits caspase-9 activation, the effect of RNA on Apaf-1 oligomerization was first analyzed using gel filtration chromatography. In unstimulated Jurkat S100 extracts, Apaf-1 was eluted on a gel filtration column as a monomer (˜160 kDa), and caspase-9 was not processed (FIG. 2A, top two panels). As expected, when treated with cytochrome c, a large portion of the Apaf-1 protein became part of the oligomeric apoptosome (˜700 kDa). At the same time, processed procaspase-9 was present both in the apoptosome-bound form and in the released form (FIG. 2A, middle two panels). In contrast, when purified total cellular RNA was added along with cytochrome c to the Jurkat S100 extracts, Apaf-1 could no longer oligomerize, and caspase-9 activation was completely blocked (FIG. 2A, bottom two panels). Therefore, RNA prevents the oligomerization of Apaf-1. It was next determined whether total RNA interferes with the binding of cytochrome c to Apaf-1. Immobilized recombinant full-length Apaf-1 readily pulled down cytochrome c in the solution. However, in the presence of RNA, the amount of cytochrome c bound to Apaf-1 was drastically decreased (FIG. 2B), suggesting that RNA inhibits the binding of cytochrome c to Apaf-1. Also examined was whether RNA has any additional effect on Apaf-1 once Apaf-1 is oligomerized. To this end, recombinant Apaf-1Δ protein (containing amino acids 1-591) was used which retained the caspase-9-binding and oligomerization domains but lacked the negative regulatory WD40 repeats that bind to cytochrome c (Riedl et al., 2005). Apaf-1Δ spontaneously forms homo-oligomers and activates caspase-9 independently of cytochrome c (Riedl et al., 2005; Srinivasula et al., 1998). As shown in FIG. 2C, total RNA had a minimal effect on Apaf-1Δ-induced caspase-9 activation. To confirm this observation, Jurkat S100 extracts were treated with cytochrome c for different periods of time and then added RNA. Treatment with cytochrome c for as little as 15 minutes rendered RNA totally ineffective in preventing caspase-9 activation (FIGS. 2D and 2E). Therefore, although RNA inhibits the interaction of cytochrome c with Apaf-1, it does not appear to directly affect subsequent events in caspase-9 activation.

The observation that RNA directly impedes the binding of cytochrome c with Apaf-1 but not the subsequent Apaf-1-involved processes suggests that the target of RNA may be cytochrome c. To test this possibility, a gel filtration assay was used to compare the size of cytochrome c in the presence or absence of total cellular RNA. Cytochrome c alone was eluted as a monomer. However, it formed higher moleCular weight complexes when incubated with total cellular RNA, suggesting that cytochrome c can directly associate with one or more cellular RNA.

### Example 3

### Cytochrome c Binds to tRNA Both In Vivo and In Vitro and Inhibits Caspase-9 Activation

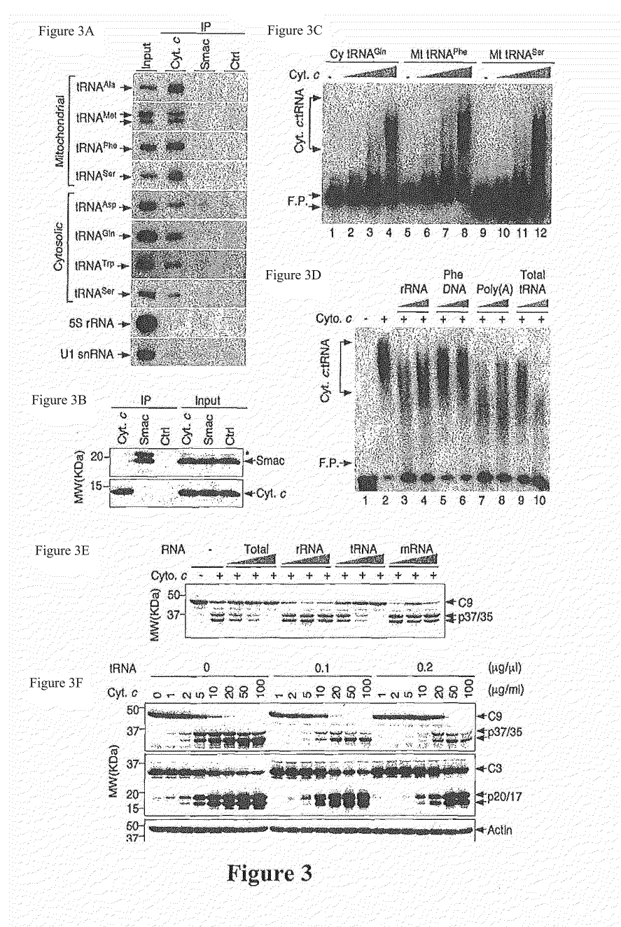

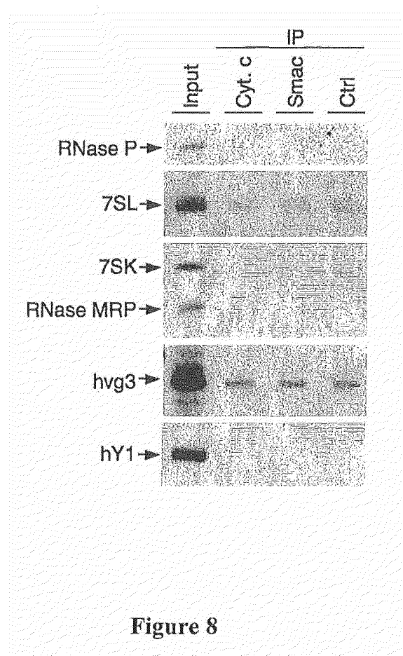

A stringent immunoprecipitation assay was used to determine whether cytochrome c or Apaf-1 specifically interacts with RNA in cells and if so which RNA species it interacts with, HeLa cells were treated with low concentrations of formaldehyde (0.2%) to cross-link RNA-protein complexes (Niranjanakumari et al., 2002). Cell lysates were then prepared in a buffer containing 1% empigen to disrupt most non-covalent post-lysis interactions (Choi and Dreyfuss, 1984). The lysates were subjected to immunoprecipitation separately with anti-cytochrome c and Apaf-1 antibodies and isotype-matching control antibodies, as well as an antibody against Smac/DIABLO. The latter, like cytochrome c, resides in the inter-membrane space of mitochondria and is released during apoptosis to participate in caspase activation (Du et al., 2000; Verhagen et al., 2000). Initial analyses showed that RNAs around 70-90 nucleotides were enriched in anti-cytochrome c immunoprecipitates but not in anti-Apaf-1, anti-Smac, or control immunoprecipitates (data not shown). Because the size of the RNA species bound to cytochrome c corresponded to that of tRNAs, Northern blot analyses were subsequently performed using specific probes for four mitochondrial and four cytoplasmic tRNAs. Probes detecting an additional eight RNA species, including several small and structured RNAs, were used as controls. Cytochrome c was associated with all four mitochondrial tRNAs in vivo. It was also associated with the four cytosolic tRNAs, although to a lesser extent (FIG. 3A, B). The specificity of tRNA:cytochrome c interaction is suggested by the absence of tRNA in anti-Smac and control immunoprecipitates as well as the lack of binding of cytochrome c to eight control RNAs (FIG. 3A and FIG. 8).

To determine whether cytochrome c directly binds to tRNA, mitochondrial tRNA^{Phe} and tRNA^{Ser} and cytosolic tRNA^{GIn} were in vitro-transcribed and -radiolabeled. These tRNAs were incubated with and without cytochrome c, and then analyzed by an electrophoretic mobility shift assay (EMSA). The mobility of each of these RNAs was slowed in the presence of cytochrome c (FIG. 3C), indicating that cytochrome c directly binds to all of the RNAs. To further characterize the cytochrome c:tRNA interaction, the binding of cytochrome c to total tRNA isolated from cells was analyzed. The mobility of the vast majority of the labeled tRNAs was slowed by cytochrome c (FIG. 3D), indicating the association of cytochrome c with various tRNAs. The binding of cytochrome c to radiolabeled tRNA could be inhibited by non-labeled total tRNA almost completely (lane 10). In contrast, the cytochrome c:tRNA binding was only partially impeded by non-labeled rRNA and poly(A), and was not affected at all by DNA. These results suggest that in vitro cytochrome c binds to RNA but not DNA, and preferentially binds to tRNA.

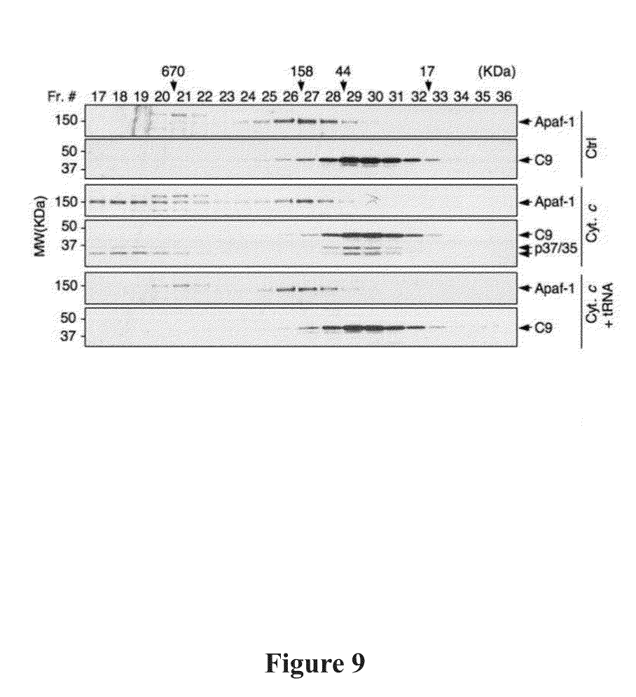

To determine the effect of tRNA on caspase-9 activation, purified cellular tRNA was added to Jurkat S100 extracts along with cytochrome c. tRNA blocked cytochrome c induced caspase-9 activation as potently as total RNA. In contrast, neither mRNA nor rRNA exhibited any significant inhibitory effect (FIG. 3D). Moreover, tRNA potently inhibited cytochrome c-induced apoptosome formation (FIG. 9). The minimal concentrations of cytochrome c that could initiate caspase-9 activation in the absence of tRNA or in the presence of two different concentrations of tRNA were compared. In the absence of tRNA, at a concentration as low as 2 μg/ml cytochrome c induced activation of caspase-9 and caspase-3. In the presence of 0.1 and 0.2 μg/μl tRNA, the minimum amount of cytochrome c required to induce caspase-9 activation increased to 10 and 20 μg/ml, respectively (FIG. 3E). This suggests tRNA may set a threshold for induction of cytochrome c-induced apoptosis.

### Example 4

### Microinjection of tRNA into Cells Inhibits Cytochrome c-Induced Apoptosis

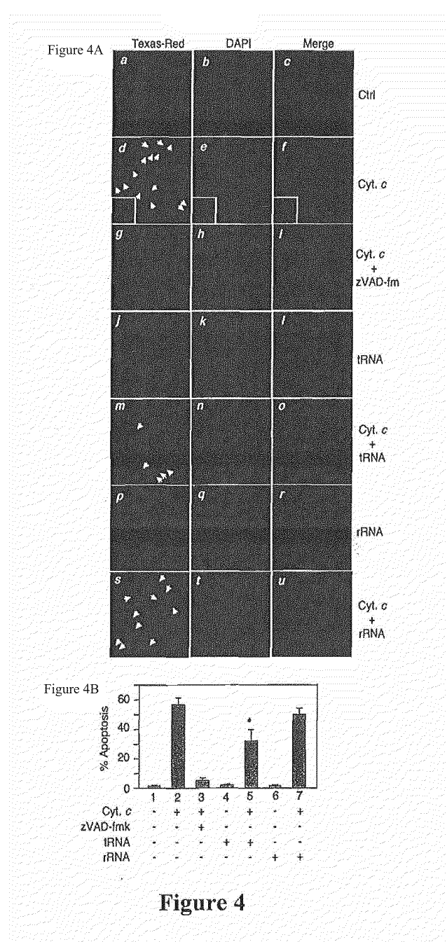

To assess the role of tRNA in cytochrome c-induced apoptosis in cells, cytochrome c was microinjected into HEK293 cells alone or together with tRNA. Microinjection of cytochrome c alone in HEK293 cells led to apoptosis in a significant number of injected cells, as determined by apoptosis-associated morphological changes such as membrane blebbing and nuclear fragmentation (Zhivotovsky et al., 1998) (Figure panels d-f and FIG. 4B, column 2). Additionally, pre-treatment of cells with the pan-caspase inhibitor zVAD-fmk nearly completely blocked apoptosis, indicating the cell death was mediated by caspases. However, when tRNA was co-injected with cytochrome c, apoptosis among injected cells noticeably decreased (FIG. 4A, panels m-o and Figure column 5). Of note, microinjection of tRNA alone had no effect on cell survival.

Furthermore, in a similar co-injection experiment, rRNA had a minimal effect on cytochrome c-induced apoptosis (FIGS. 4A and 4B). Therefore, cytochrome c-mediated apoptosis is specifically inhibited by tRNA.

### Example 5

### Degradation of Cellular tRNA Enhances Apoptosis Via the Intrinsic Pathway

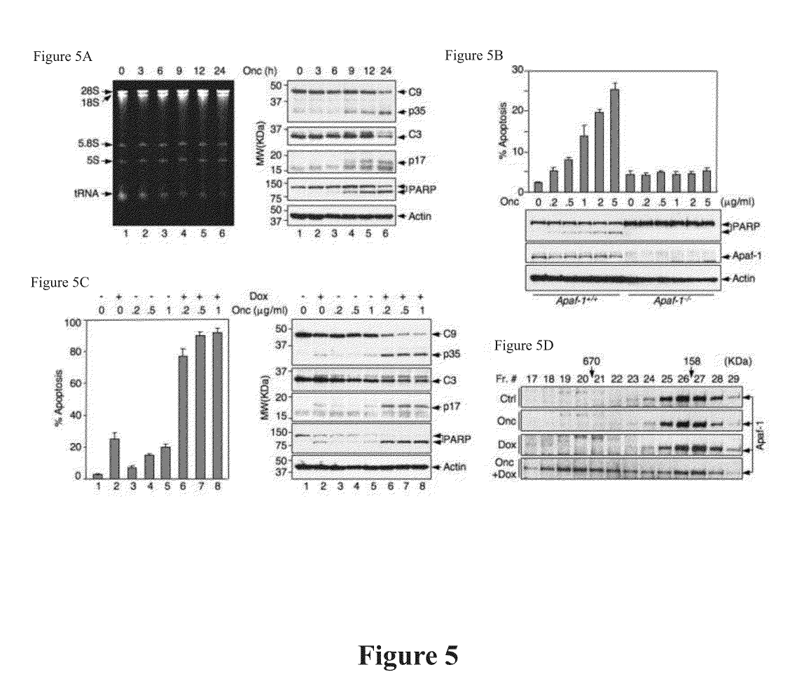

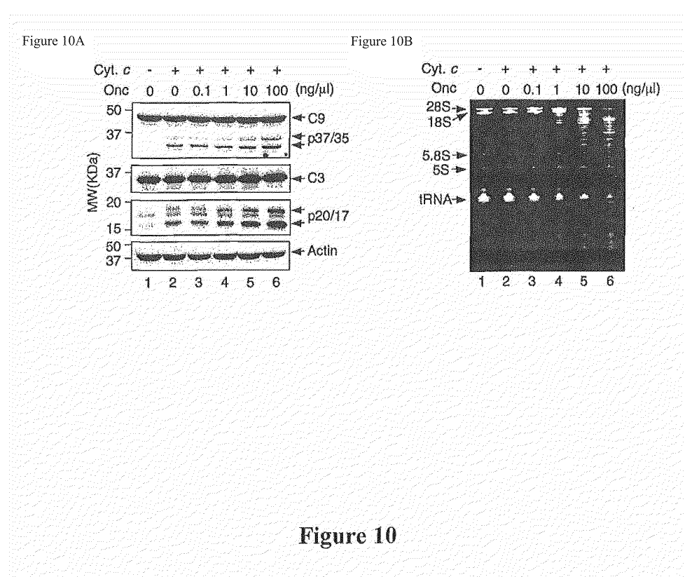

To investigate the effect of tRNA hydrolysis on caspase-9 activation and apoptosis in cells, the tRNA-specific ribonuclease onconase (ranpirnase) was used. Consistent with previous observations (Iordanov et al., 2000; Saxena et al., 2002; Suhasini and Sirdeshmukh, 2006), transfection of onconase into HeLa cells resulted in the degradation of tRNA, but not various rRNAs (FIG. 5A, left panel). The degradation of tRNA was closely followed by the activation of caspase-9 and -3 and the cleavage of the apoptotic substrate PARP (FIG. 5A, right panels), suggesting that tRNA degradation could determine onconase-induced apoptosis. In HeLa S 100 extracts, treatment with onconase also enhanced cytochrome c-induced caspase activation in a dosage-dependent manner (FIG. 10). To confirm that onconase-induced apoptosis was dependent on Apaf-1, Apaf-1-deficient mouse embryonic fibroblasts (MEFs) and wild type MEFs were treated with onconase. Onconase killed Apaf-1^{-/-} MEFs but not wild type MEFs (FIG. 5B).

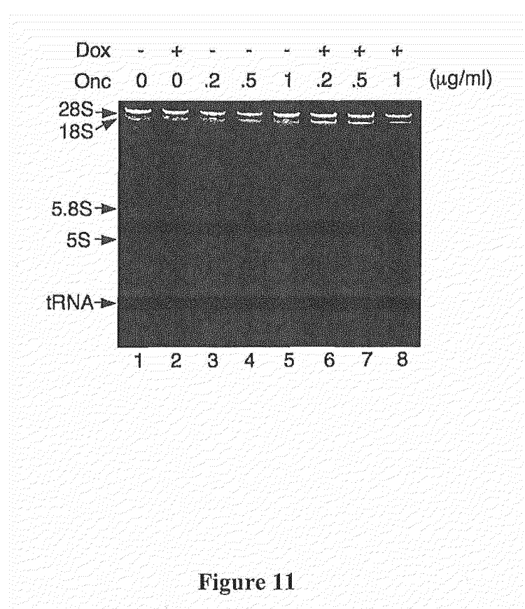

If onconase promotes apoptosis by reversing the inhibitory effect of tRNA on cytochrome c-induced apoptosome formation, it should enhance cell death stimulated by agents invoking the intrinsic apoptosis pathway. This possibility was tested by examining the individual and combined effect of onconase and the genotoxic drug doxorubicin on apoptosis induction. At dosages that alone induced substantial tRNA degradation but caused only minimal levels of caspase activation and apoptosis, onconase enhanced doxorubicin-induced apoptosis from 30% to over 95% (FIG. 5C and FIG. 11). A gel-filtration analysis showed that onconase and doxorubicin together, but not individually, promoted the assembly of Apaf-1 into the apoptosome (FIG. 5D). These results show that degradation of cellular tRNA enhances apoptosis via the intrinsic pathway.

Since its discovery nearly 50 years ago, tRNA has been studied almost exclusively in the context of the flow of genetic information, as the adaptor between codons and amino acids in protein translation or a primer for reverse transcription (Hopper and Shaheen, 2008; Ibba et al., 2000; Weisblum, 1999). The data herein provide evidence that tRNA may have an unexpected role beyond gene expression. By binding to cytochrome c and preventing its association with Apaf-1, tRNA directly promotes cell survival. This finding likely reveals an intimate connection between tRNA and cytochrome c, linking the protein translation pathway to cell death. This finding may have direct implications in cancer therapy.

It is notable that cytochrome c interacts with mitochondrial tRNA in healthy cells (FIG. 3A). Mitochondria contain several other apoptosis inducers in addition to cytochrome c, such as Smac/Diablo, EndoG, and AIF (Riedl and Salvesen, 2007; Wang, 2001). This is the first evidence that mitochondria also harbor at least one antidote. This inhibitory effect of mitochondrial tRNA on the pro-apoptotic function of cytochrome c might reflect the need to tightly regulate cytochrome c when it first acquired such a destructive power. Cytosolic tRNA is also capable of associating with cytochrome c in healthy cells (FIG. 3A). This is consistent with a recent report that mammalian cytosolic tRNA can be efficiently imported into mitochondria (Rubio et al., 2008). Unlike mitochondria in some other organisms, which rely on cytosolic tRNA for protein synthesis, mammalian mitochondria encode the complete set of tRNAs for their own protein synthesis. The function of cytosolic tRNA in mitochondria is thus unclear. An intriguing possibility is that cytosolic tRNA transferred into the mitochondria may help coordinate the rate of protein synthesis in the cytosol with cellular resistance to apoptosis stimuli targeted to mitochondria. tRNA in the cytosol could then further block apoptosis once cytochrome c is released from mitochondria; this is supported by the effect of tRNA microinjection and hydrolysis on cytochrome c-induced apoptosis (FIG. 4).

Cytochrome c appears to interact with tRNA but not other RNAs, especially in vivo. The reason for this specific binding remains to be determined, but it could be a combination of intrinsic preference of cytochrome c to tRNA and the relative accessibility of tRNA compared with other RNAs in cells. Notably, cytochrome c has a high content of lysines and arginines, somewhat similar to ancillary RNA-binding domains present in the N- and C-termini of certain eukaryotic amino acid-tRNA synthetases (Cahuzac et al., 2000; Kaminska et al., 2001).

In mammalian cells, transcription of tRNA by Pol III is stimulated by oncogenic proteins Myc and Erk and inhibited by tumor suppressors RB and p53 (Ruggero and Pandolfi, 2003; White, 2005). Tumor cells synthesize tRNA at highly enhanced rates due to the deregulation of these tumor suppressors and oncoproteins, and in some cases, also due to direct elevation in the expression of the pol III factor TFIIIC (White, 2004; White, 2005). Suppression of apoptosis may be an important part of the transforming activity of tRNA. Notably, the tRNA-specific RNase onconase kills tumor cells with relatively low systemic toxicity (Ardelt et al., 2008; Costanzi et al., 2005). Onconase also has the advantage of p53 independent killing (Costanzi et al., 2005; Schein, 1997), important as many tumors, especially those resistant to radiation and chemotherapies, lack p53 activity. Onconase is in phase III clinical trials for the treatment of mesothelioma, a specific lung cancer, and phase II clinical trials for other cancers (Ardelt et al., 2008; Costanzi et al., 2005). The reasons for tumor-specific toxicity of onconase is not clear. The effect of onconase on a critical inhibitor of apoptosis may not only provide an explanation but also indicate a valuable target for therapeutic intervention. The synergistic action of onconase with agents that elicit the intrinsic apoptosis pathway (FIG. 5D) provides a biologic rationale for development of combined cancer therapies.

### Example 6

### Characterization of Cytochrome c and Transfer RNA Interaction

The results presented herein demonstrate that multiple transfer RNA (tRNA) species bind cytochrome c in the cell and that cellular tRNA is capable of inhibiting cytochrome c-mediated apoptosis. Experiments were designed to extend these findings to better define the structural basis of the interaction of tRNA with cytochrome c and which tRNA species associate with cytochrome c in the cell. Experiments were also designed to explore ways in which tRNA may affect cytochrome c function and the significance this relations in apoptosis and metabolism.

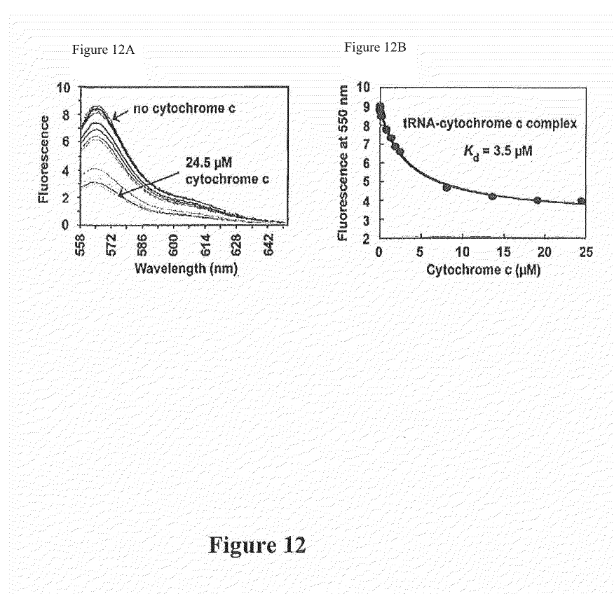

The strength and specificity of cytochrome c and tRNA interaction was assessed using two methods. It was observed that cytochrome c addition quenches florescence from a synthesized 5′ Cy-3 tagged tRNACys probe. Analysis with a hyperbolic equation obtained a Kd of 3.5 μM for the binding interaction (FIG. 12). This is similar to Kd values of tRNA interactions with aminoacyl-tRNA synthetases, tRNA-methyl transferases, and CCA-adding enzymes.

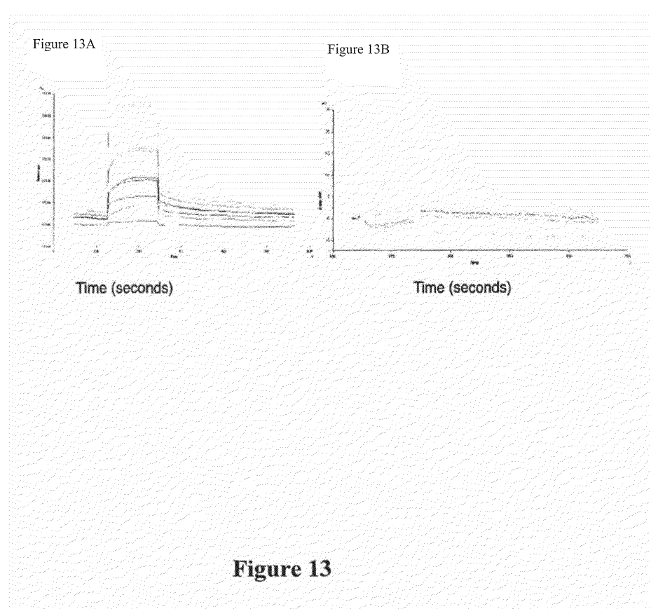

The affinity of native tRNA for cytochrome c was also tested using surface Plasmon resonance using a Biacore 3000 system. Cytochrome c was immobilized on a CMS sensor chip (Biacore) by amine coupling, and transfer RNA from bovine liver, ribosomal RNA from bovine liver, polyadenylic acid (Sigma) and a 70 bp DNA oligo with a phenylalanine tRNA sequence (IDT) were individually injected onto immobilized cytochrome c at 50 ng/ml in physiologic salt concentration. Only transfer RNA bound above baseline (FIG. 13).

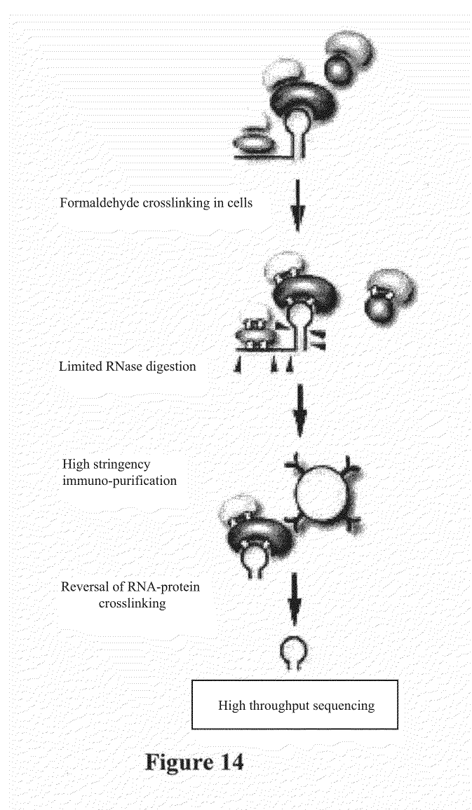

The next series of experiments were designed to use a high-throughput sequencing strategy to identify RNA directly associated with cytochrome c (FIG. 14). HeLa cells were cross-linked in strong detergent and immunoprecipitated with anti-cytochrome c antibody and performed limited digestion with RNase. RNAs were released by heat and RNA fragments were ligated to 5′ and 3′ linkers, reversed transcribed, and amplified by PCR to construct a cDNA library. The library was subjected to high throughput sequencing with an Illumina Genome Analyzer.

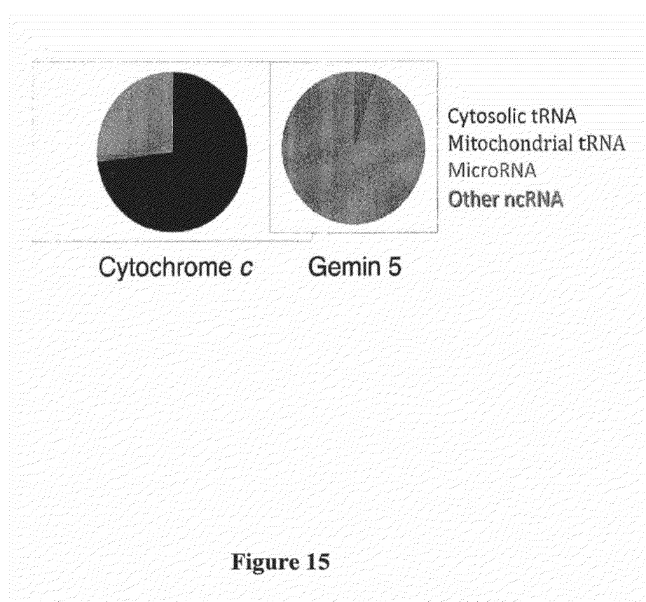

Approximately 50,000 sequence reads could be mapped to non-coding RNAs on the UCSC genome browser. tRNAs were the major non-rRNA non-coding RNA component discovered. This was especially striking when compared to a recent result with the same protocol in the same cells but a different antibody against GeminS, a component of the survival of motor neurons complex) (FIG. 15). Both cytoplasmic and mitochondrial tRNAs were enriched substantially, and in each group there was a very large relative frequency range between different tRNAs.

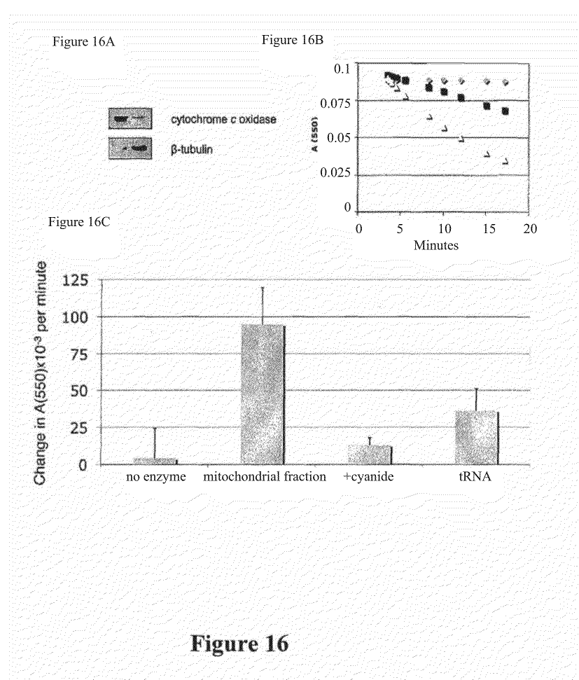

Based on the association in healty cells, it is believed that tRNA may affect the function of cytochrome c in oxidative phosphorylation. It was tested whether tRNA affected oxidation of reduced cytochrome c by the electron transport enzyme cytochrome c oxidase. Cytochrome c from bovine heart (Sigma) was treated with ascorbic acid and purified reduced cytochrome c on a sepharose column. Crude mitochondrial extracts were purified from 293T cells by differential centrifugation and permeabilized freeze-thaw. Addition of tRNA at concentrations ˜1:1 relative to cytochrome c inhibited the rate of cytochrome c oxidation substantially (FIG. 16). This result is consistent with the notion that tRNA may inhibit oxidative phosphorylation by binding to cytochrome c.

The results presented herein support a specific high-affinity interaction between tRNA and cytochrome c in human cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### REFERENCES

Ardelt et al., (2008). Curr Pharm Biotechnol 9, 215-225.
Bao et al., 2007). Mol Cell 25, 181-192.
Cahuzac et al., (2000). Embo J 19, 445-452.
Chandra et al., (2006). Cell 125, 1333-1346.
Chang et al., (2003). J Biol Chem 278, 16466-16469.
Chang et al., (2000). Microbiol Mol Biol Rev 64, 821-846.
Choi et al., (1984). J Cell Biol 99, 1997-1204.
Costanzi et al., (2005). Cancer Invest 23, 643-650.
Du et al., (2000). Cell 102, 33-42.
Hopper et al., (2008). Trends Cell Biol 18, 98-104.
Ibba et al., 2000 Trends Biochem Sci 25, 311-316.
Iordanov et al., (2000). Cancer Res 60, 1983-1994.
Jiang et al., (2000). J Biol Chem 275, 31199-31203.
Kaminska et al., (2001). Biochemistry 40, 14309-14316.
Kim et al., (2005) Proc Natl Acad Sci USA 102, 17545-17550.
Li et al., (2008). Caspases Oncogene 27, 6194-6206.
Li et al., (1997). Cell 91, 479-489.
Liu et al., (2005). J Biol Chem 280, 11578-11582.
Liu et al., (1996). Cell 86, 147-157.
Marshall et al., (2008). Cell 133, 78-89.
Niranjanakumari et al., (2002). Methods 26, 182-190.
Riedl et al., (2005). Nature 434, 926-933.
Riedl et al., (2007). Nat Rev Mol Cell Biol 8, 405-413.
Riedl et al., (2004). Nat Rev Mol Cell Biol 5, 897-907.
Rubio et al., (2008). Proc Natl Acad Sci USA 105, 9186-9191.
Ruggero et al., (2003). Nat Rev Cancer 3, 179-192.
Saxena et al., (2002). J Biol Chem 277, 15142-15146.
Schafer et al., (2006). Dev Cell 10, 549-561.
Schein et al., (1997). Nat Biotechnol 15, 529-536.
Srinivasula et al., (1998). Mol Cell 1, 949-957.
Suhasini et al., (1995). Science267, 1456-1462.
Vaux et al., (1999). Cell 96, 245-254.
Verhagen et al., (2000). Cell 102, 43-53.
Wang et al., (2001). Genes Dev 15, 2922-2933.
Weisblum et al., (1999). Trends Biochem Sci 24, 247-250.
White et al., (2004). Oncogene 23, 3208-3216.
White et al., (2005). Nat Rev Mol Cell Biol 6, 69-78.
Zhivotovsky et al., (1998). Nature 391, 449-450.
Zou, et al., (1997). Cell 90, 405-413.
Zou, et al., (1999). J Biol Chem 274, 11549-11556.

What is claimed:

1. A method of enhancing survival of a cell, said method comprising inhibiting the formation of an apoptosome in a cell by contacting said cell with an effective amount of a tRNA activator, wherein when said tRNA activator contacts said cell, said tRNA activator increases the expression, function, stability, or activity of said tRNA, wherein said tRNA binds to cytochrome c, thereby enhancing survival of said cell.

2. The method of claim 1, wherein said cell is a mammalian cell.

3. The method of claim 2, wherein said cell is a human cell.

4. A method of inhibiting survival of a cell, said method comprising enhancing formation of an apoptosome in a cell by contacting said cell with an effective amount of a tRNA inhibitor, wherein when said tRNA inhibitor contacts said cell, said tRNA inhibitor decreases expression, function, stability, or activity of said tRNA, wherein said tRNA does not bind to cytochrome c, thereby inhibiting survival of said cell.

5. The method of claim 4, wherein said tRNA inhibitor is selected from the group consisting of a protein, a peptide, an siRNA, a ribozyme, an antisense, an aptamer, a peptidomimetic, a small molecule, or any combination thereof.

6. The method of claim 4, wherein said cell is a mammalian cell.

7. The method of claim 6, wherein said mammalian cell is a human cell.

8. The method of claim 7, wherein said human cell is a cancer cell.

9. The method of claim 5, wherein said protein is an RNase.

10. The method of claim 9, wherein said RNase is onconase.

11. The method of claim 4, wherein said tRNA inhibitor is administered in combination with a therapeutically effective amount of another therapeutic agent.

12. The method of claim 11, wherein said therapeutic agent is doxorubicin.

13. A method of augmenting tRNA expression, function or activity in a cell, said method comprising contacting said cell with a tRNA activator, wherein when said tRNA activator contacts said cell, said tRNA activator augments said tRNA expression, function, or activity in said cell, wherein said tRNA does not bind to cytochrome c, thereby inhibiting survival of said cell.

14. The method of claim 13, wherein said tRNA activator is selected from the group consisting of a protein, a peptide, an siRNA, a ribozyme, an antisense, an aptamer, a peptidomimetic, a small molecule, or any combination thereof.

15. The method of claim 13, wherein said cell is a mammalian cell.

16. The method of claim 15, wherein said mammalian cell is a human cell.

17. A method of inhibiting tRNA expression, function or activity in a cell, said method comprising contacting a cell with a tRNA inhibitor, wherein when said tRNA inhibitor contacts said cell, said tRNA inhibitor reduces said tRNA expression, function, or activity in said cell, wherein said tRNA does not bind cytochrome c, thereby inhibiting survival of said cell.

18. The method of claim 17, wherein said tRNA inhibitor is selected from the group consisting of a protein, a peptide, an siRNA, a ribozyme, an antisense, an aptamer, a peptidomimetic, a small molecule, or any combination thereof.

19. The method of claim 17, wherein said cell is a mammalian cell.

20. The method of claim 19, wherein said cell mammalian cells is a human cell.

21. A method of inhibiting an interaction between cytochrome c and Apaf-1 in a cell, said method comprising contacting said cell with an effective amount of a tRNA activator, wherein said tRNA activator increases tRNA expression, activity, stability, or function in said cell, thereby inhibiting said interaction between cytochrome c and Apaf-1 and enhancing cell survival.

22. The method of claim 21, wherein said tRNA activator is selected from the group consisting of a protein, a peptide, an siRNA, a ribozyme, an antisense, an aptamer, a peptidomimetic, a small molecule, or any combination thereof.

23. The method of claim 21, wherein said cell is a mammalian cell.

24. The method of claim 23, wherein said mammalian cell is a human cell.

25. A method of increasing an interaction between cytochrome c and Apaf-1 in a cell, said method comprising contacting said cell with an effective amount of a tRNA inhibitor, wherein said tRNA inhibitor increases tRNA expression, activity, stability, or function in said cell, thereby increasing said interaction between cytochrome c and Apaf-1, thereby decreasing cell survival.

26. The method of claim 25, wherein said tRNA inhibitor is selected from the group consisting of a protein, a peptide, an siRNA, a ribozyme, an antisense, an aptamer, a peptidomimetic, a small molecule, or any combination thereof.

27. The method of claim 25, wherein said cell is a mammalian cell.

28. The method of claim 27, wherein said cell is a human cell.

29. A method of treating a disease associated with aberrant cytochrome c release in a mammal, the method comprising administering to a mammal in need thereof a composition comprising a tRNA activator or a tRNA inhibitor.


SEQUENCE LISTING (25 sequences)

SEQ ID NO: 1 - DNA, 69 bp, Homo sapiens
aagggcttagcttaattaaagtggctgatttgcgttcagttgatgcagagtggggttttgcagtcctta

SEQ ID NO: 2 - DNA, 71 bp, Homo sapiens
acttttaaaggataacagctatccattggtcttaggccccaaaaattttggtgcaactccaaataaaagta

SEQ ID NO: 3 - DNA, 68 bp, Homo sapiens
agtaaggtcagctaaataagctatcgggcccataccccgaaaatgttggttatacccttcccgtacta

SEQ ID NO: 4 - DNA, 71 bp, Homo sapiens
gtttatgtagcttacctcctcaaagcaatacactgaaaatgtttagacgggctcacatcaccccataaaca

SEQ ID NO: 5 - DNA, 59 bp, Homo sapiens
gagaaagctcacaagaactgctaactcatgcccccatgtctaacaacatggctttctca

SEQ ID NO: 6 - DNA, 72 bp, Homo sapiens
ggttccatggtgtaatggtaagcactctggactctgaatccagccatctgagttcgagtctctgtggaacct

SEQ ID NO: 7 - DNA, 82 bp, Homo sapiens
gtagtcgtggccgagtggttaaggcgatggactagaaatccattggggtctccccgcgcaggttcgaatcctgccgactacg

SEQ ID NO: 8 - DNA, 72 bp, Homo sapiens
gacctcgtggcgcaacggtagcgcgtctgactccagatcagaaggttgcgtgttcaaatcacgtcggggtca

SEQ ID NO: 9 - DNA, 72 bp, Homo sapiens
tccttgttactatagtggtgagtatctctgcctgtcatgcgtgagagagggggtcgattccccgacggggag

SEQ ID NO: 10 - DNA, 60 bp, Homo sapiens
tgcaaaaccccactctgcatcaactgaacgcaaatcagccactttaattaagctaagccc

SEQ ID NO: 11 - DNA, 60 bp, Homo sapiens
tacgggaagggtataaccaacattttcggggtatgggcccgatagcttatttagctgacc

SEQ ID NO: 12 - DNA, 60 bp, Homo sapiens
ggggtgatgtgagcccgtctaaacattttcagtgtattgctttgaggaggtaagctacat

SEQ ID NO: 13 - DNA, 30 bp, Homo sapiens
agctggtttcaagccaaccccatggcctcc

SEQ ID NO: 14 - DNA, 60 bp, Homo sapiens
cggggaatcgaccccctctctcacgcatgacaggcagagatactcaccactatagtaaca

SEQ ID NO: 15 - DNA, 30 bp, Homo sapiens
ttcagagtccagagtgcttaccattacacc

SEQ ID NO: 16 - DNA, 60 bp, Homo sapiens
acctgcgcggggagaccccaatggatttctagtccatcgccttaaccactcggccacgac

SEQ ID NO: 17 - DNA, 25 bp, Homo sapiens
caccttcgtgatcatggtatctccc

SEQ ID NO: 18 - DNA, 25 bp, Homo sapiens
caccttcgtgatcatggtatctccc

SEQ ID NO: 19 - DNA, 25 bp, Homo sapiens
cctacagcacccggtattcccaggc

SEQ ID NO: 20 - DNA, 25 bp, Homo sapiens
tgcacgtggcactctctgcccgagg

SEQ ID NO: 21 - DNA, 25 bp, Homo sapiens
tcctgcccagtctgacctcgcgcgg

SEQ ID NO: 22 - DNA, 30 bp, Homo sapiens
cgcctagccagccagatcagccgaatcaac

SEQ ID NO: 23 - DNA, 30 bp, Homo sapiens
acccctccttaggcaacctggtggtccccc

SEQ ID NO: 24 - DNA, 20 bp, Homo sapiens
agaggtggtttgatgacacg

SEQ ID NO: 25 - DNA, 30 bp, Homo sapiens
tgaacaatcaattgagataactcactacct